(19) United States
(12) Patent Application Publication
(10) Pub. No.: US 2023/0321353 A1
(43) Pub. Date: Oct. 12, 2023

(54) DRUG CARTRIDGE, CASSETTE, DRUG INJECTION DEVICE, AND DRUG INJECTION SYSTEM

(71) Applicant: PHC Holdings Corporation, Tokyo (JP)

(72) Inventors: Yuri ENDO, Ehime (JP); Hisanori KATOH, Ehime (JP); Shinsuke HATA, Ehime (JP); Takashi SHIRAKAWA, Ehime (JP); Kazumasa OKAMURA, Ehime (JP); Takuya SUZUKI, Ehime (JP); Tadashi MATSUMOTO, Ehime (JP); Kazuho OMOTE, Ehime (JP); Ryosuke TANI, Ehime (JP); Noriyuki SHINOHARA, Ehime (JP)

(21) Appl. No.: 18/023,230

(22) PCT Filed: Nov. 24, 2021
(86) PCT No.: PCT/JP2021/043006

§ 371 Date: Feb. 24, 2023

(30) Foreign Application Priority Data
Dec. 2, 2020 (JP) 2020-199996

(51) Int. Cl. A61M 5/315; A61M 5/20

(52) U.S. Cl. CPC A61M 5/20; A61M 5/31511; A61M 5/31585; G16H 20/17

(57) ABSTRACT

A drug cartridge includes: a cylinder 11 having a cylindrical cylinder columnar space extending in a longitudinal direction; a gasket 13 supported in the space so that the gasket is movable in the longitudinal direction; a drug 14 held in the space and at least including a liquid first component; and a first temperature sensor and an RF tag 16 arranged on a side surface of the cylinder. The RF tag 16 stores drug information that includes at least information indicating a type of the drug, and wirelessly transmits at least the information indicating the type of the drug and first temperature information indicating a temperature detected by the first temperature sensor in response to an instruction from outside.

### TECHNICAL FIELD

The present application relates to a drug cartridge for medical use, a cassette housing the same, a drug injection device, and a drug injection system.

### BACKGROUND ART

Patients suffering from certain diseases may be prescribed, for example, to inject a drug such as insulin or growth hormone several times a day. In order for patients to inject such a drug by themselves (also called self-injection), various drug injection devices have been put into practical use, as disclosed in Patent Document 1, etc.

### CITATION LIST

### Patent Literature

Patent Document No. 1: Japanese National Phase PCT Laid-open Publication No. 2014-516634

### SUMMARY OF INVENTION

### Technical Problem

A drug cartridge may contain a quantity of drug that is injected in multiple doses depending on the specifications or the prescription. In this case, it is preferred to properly manage the drug. Performing multiple injections each day may be bothersome to the patient from various perspectives. In view of this, the present application provides a drug cartridge, a cassette, a drug injection device, and a drug injection system, with which it is possible to properly manage the drug and the injection, or reduce the burden on the operator.

### Solution to Problem

A drug cartridge according to an embodiment of the present disclosure includes: a cylinder having a cylindrical cylinder columnar space extending in a longitudinal direction; a gasket supported in the space so that the gasket is movable in the longitudinal direction; a drug held in the space and at least including a liquid first component; and a first temperature sensor and an RF tag arranged on a side surface of the cylinder, wherein the RF tag stores drug information that includes at least information indicating a type of the drug, and wirelessly transmits at least the information indicating the type of the drug and first temperature information indicating a temperature detected by the first temperature sensor in response to an instruction from outside.

A drug injection device according to an embodiment of the present disclosure includes: a device housing having a housing space that houses at least a portion of a cassette that houses a drug cartridge having a first temperature sensor and an RF tag, and a housing opening that communicates with the housing space; a piston supported so that the piston can move into the housing space; a motor configured to drive the piston; a motor driver configured to generate a drive signal for driving the motor; an antenna arranged adjacent to the housing space; a transmission/reception circuit configured to transmit electromagnetic waves from the antenna and receive electromagnetic waves received by the antenna; a display device configured to output information regarding an injection operation; and a control device configured to control the motor driver, the transmission/reception circuit and the display device, wherein: with the drug cartridge loaded in the housing space, the control device causes the transmission/reception circuit to receive, via the antenna, drug information including information indicating a type of a drug in the drug cartridge transmitted from an RF tag of the drug cartridge and first temperature information detected by the first temperature sensor, and determines a drive power of the motor based on the first temperature information to control the motor driver so as to output the determined drive power.

### Advantageous Effects of Invention

The present disclosure provides a drug injection device that can be properly managed.

### BRIEF DESCRIPTION OF DRAWINGS

FIG. 1 is a perspective view showing a drug injection system including a cassette, a drug injection device and a charger.

FIG. 2(a), FIG. 2(b) and FIG. 2(c) are perspective views illustrating how a drug cartridge is housed in the cassette.

FIG. 3(a), FIG. 3(b) and FIG. 3(c) are perspective views illustrating how the cassette is housed in the drug injection device.

FIG. 4(a) and FIG. 4(b) are perspective views illustrating the state where injection is performed.

FIG. 5 is a block diagram showing an example configuration of an electrical circuit of the drug injection device.

FIG. 6A is a view illustrating how the drug injection device is set in a charger.

FIG. 6B is a front view showing the drug injection device set in the charger.

FIG. 7A is a cross section parallel to the longitudinal direction of the drug cartridge.

FIG. 7B is a cross section perpendicular to the longitudinal direction of the drug cartridge.

FIG. 8A is a schematic plan view of an RF tag.

FIG. 8B is a schematic plan view of another RF tag.

FIG. 9A shows a schematic cross section of another drug cartridge.

FIG. 9B shows a schematic cross section of the other drug cartridge taken along line 9B-9B of FIG. 9A.

FIG. 10(a) and FIG. 10(b) are schematic diagrams illustrating the movement of a liquid component.

FIG. 11 is an exploded perspective view of a hood of the cassette.

FIG. 12(a) and FIG. 12(b) are views illustrating how a needle unit is attached to the cassette.

FIG. 13 is an exploded perspective view of the drug injection device with the device housing removed.

FIG. 14 is a perspective view of the drug injection device, showing the arrangement of an RF-ID antenna.

FIG. 15 is an exploded perspective view of a piston drive mechanism.

FIG. 16A is a schematic diagram showing the positional relationship between the RF tag of the drug cartridge and the antenna in the drug injection device.

FIG. 16B is a schematic diagram showing the positional relationship between the RF tag of the drug cartridge and the antenna in the drug injection device.

FIG. 17 shows an example of drive information by PWM.

FIG. 18A shows an example of a starting current that occurs when a PWM control is not performed.

FIG. 18B shows an example of a starting current by a PWM control of the present embodiment.

FIG. 19A is a flow chart illustrating the operation of the drug injection device during an injection operation.

FIG. 19B is a flow chart illustrating the operation of the drug injection device during an injection operation.

FIG. 19C is a flow chart illustrating a remind operation.

FIG. 20 shows an example of a detection result of a touch sensor.

FIG. 21 is a flow chart illustrating the injection operation of the drug injection device using the touch sensor.

FIG. 22 is a schematic diagram illustrating the orientations of the three axes of an acceleration sensor.

FIG. 23 is a schematic diagram illustrating a portion where it is difficult to remove air during air removal.

FIG. 24 is a schematic diagram illustrating the attitude suitable for air removal.

FIG. 25 is a flow chart illustrating an air-removing operation using an acceleration sensor.

FIG. 26 is a flow chart illustrating a mixing operation.

FIG. 27 is a schematic diagram showing zones used in the mixing operation.

FIG. 28 is a schematic diagram illustrating an air-removing operation.

FIG. 29 is an exploded perspective view showing a portion of a piston drive mechanism including a rotary encoder.

FIG. 30 is a plan view of an encoder plate.

FIG. 31 shows an example of a pulse signal.

FIG. 32 is a block diagram showing an example of a fault detector.

FIG. 33 shows an example image showing information stored in an RF tag.

FIG. 34(a) to FIG. 34(c) show example images to be displayed in an air-removing operation.

FIG. 35(a) and FIG. 35(b) show example images prompting the operator to maintain the same state until completion of an operation of the drug injection device.

FIG. 36(a) to FIG. 36(f) show example images prompting to attach a needle unit and to remove a needle case.

FIG. 37(a) to FIG. 37(f) show example images to be displayed during injection.

FIG. 38A is a front view showing how a drug cartridge is loaded in the drug injection device.

FIG. 38B is a front view showing how a drug cartridge is loaded in the drug injection device.

FIG. 38C is a front view showing how a drug cartridge is loaded in the drug injection device.

FIG. 39 is views illustrating how a needle unit is attached to, or a used injection needle is removed from, the drug cartridge loaded in the drug injection device.

FIG. 40 is views illustrating how a needle unit is attached to, or a used injection needle is removed from, the drug cartridge loaded in the drug injection device.

FIG. 41 is views illustrating how a needle unit is attached to, or a used injection needle is removed from, the drug cartridge loaded in the drug injection device.

FIG. 42 is views illustrating how an injection is performed using the drug injection device.

### DESCRIPTION OF EMBODIMENTS

When a drug cartridge contains multiple doses of a drug, it is preferred that the drug cartridge or drug injection device that is in the process of use and still has some drug remaining in it is stored in a cold place, such as a refrigerator, to control drug deterioration. However, when a drug is stored in a cold place, the viscosity of the drug increases due to the low temperature. Therefore, if one takes the drug cartridge or drug injection device out of the cold place and immediately uses it for injection, there is a need to eject the highly viscous drug from a small-diameter injection needle, which places a heavy load on the injection motor, etc., making it difficult to appropriately control the drive of the motor. Moreover, some patients feel pain when a low-temperature drug is injected.

For this reason, it is generally recommended that the drug cartridge or drug injection device be left at room temperature for some tens of minutes after being taken out of the cold place, and that the injection be performed after the temperature of the drug has risen to about room temperature. This could solve the problem described above.

However, it can be said that the wait time before injection is substantially part of the time required for a single injection, and it is therefore preferred for the patient that the wait time be short. Moreover, it is preferred that the pain of injection be small. On the other hand, it is preferred that the drug injection device be appropriately controlled in accordance with the viscosity of the drug.

In view of such problems, the present inventors have conceived of a drug cartridge, a cassette, a drug injection device, and a drug injection system that can be properly managed and reduce the burden on the operator. The drug cartridge, the cassette, the drug injection device and the drug injection system of the present disclosure are as follows.

[Item 1] A drug cartridge including:
a cylinder having a cylindrical cylinder columnar space extending in a longitudinal direction;
a gasket supported in the cylinder columnar space so that the gasket is movable in the longitudinal direction;
a drug held in the cylinder columnar space and at least including a liquid first component; and
a first temperature sensor and an RF tag arranged on a side surface of the cylinder, wherein:
   the RF tag stores drug information that includes at least information indicating a type of the drug, and wirelessly transmits at least the information indicating the type of the drug and first temperature information indicating a temperature detected by the first temperature sensor in response to an instruction from outside.

[Item 2] The drug cartridge according to item 1, wherein the RF tag is of a passive type.

[Item 3] The drug cartridge according to item 1, wherein the RF tag further stores at least one of information indicating an expiration date of the drug and information indicating an initial amount of the drug.

[Item 4] A cassette housing a drug cartridge that is loaded into a drug injection device, the cassette including:
a cassette body having a cassette columnar space capable of housing at least a portion of the drug cartridge, an injection needle attachment portion that is located at a distal end of the cassette columnar space and to/from which an injection needle can be attached/detached, and a body opening located at a rear end of the cassette columnar space and accessible to the cassette columnar space;
a cassette cap supported in the vicinity of the rear end of the cassette body so as to open/close the body opening; and
a hood including a hood body supported by the cassette body so that the hood body can pivot between a first position at which the injection needle attachment portion of the cassette body is covered and a second position at which the injection needle attachment portion is exposed, a needle covering supported by the hood body so that the needle covering can move between a projecting position at which the needle covering projects relative to the hood body and a stowed position at which the needle covering is at least partially stowed, and a biasing member configured to bias the needle covering in a direction of the projecting position.

[Item 5] The cassette according to item 4, wherein a portion of the needle covering is transparent.

[Item 6] The cassette according to item 5, wherein the needle covering includes a transparent first portion and a semi-transparent second portion.

[Item 7] The cassette according to any one of items 4 to 6, wherein the hood includes an arm that extends on an opposite side of the hood body relative to a fulcrum of the pivot and is connected to the hood body.

[Item 8] A drug injection device, including:
a device housing having a housing space that houses at least a portion of a cassette that houses a drug cartridge having a first temperature sensor and an RF tag, or at least a portion of a drug cartridge having a first temperature sensor and an RF tag and being not housed in the cassette, and a housing opening that communicates with the housing space;
a piston supported so that the piston can move into the housing space;
a motor configured to drive the piston;
a motor driver configured to generate a drive signal for driving the motor;
an antenna arranged adjacent to the housing space;
a transmission/reception circuit configured to transmit electromagnetic waves from the antenna and receive electromagnetic waves received by the antenna;
a display device configured to output information regarding an injection operation; and
a control device configured to control the motor driver, the transmission/reception circuit and the display device, wherein:
   with the drug cartridge loaded in the housing space, the control device causes the transmission/reception circuit to receive, via the antenna, drug information including information indicating a type of a drug in the drug cartridge transmitted from an RF tag of the drug cartridge and first temperature information detected by the first temperature sensor, and determines a drive power of the motor based on the first temperature information to control the motor driver so as to output the determined drive power.

[Item 9] A drug injection device, including:
a device housing having a housing space that houses at least a portion of a cassette that houses a drug cartridge having a first temperature sensor and an RF tag, or at least a portion of a drug cartridge having a first temperature sensor and an RF tag and being not housed in the cassette, and a housing opening that communicates with the housing space;
a piston supported so that the piston can move into the housing space;
a motor configured to drive the piston;
a motor driver configured to generate a drive signal for driving the motor;
an antenna arranged adjacent to the housing space;
a transmission/reception circuit configured to transmit electromagnetic waves from the antenna and receive electromagnetic waves received by the antenna;
a memory storing control information for controlling the motor for each of a plurality of drugs, wherein the control information of each drug includes a set of control parameters of the motor for each of a plurality of temperature ranges;
a display device configured to output information regarding an injection operation; and
a control device configured to control the motor driver, the transmission/reception circuit, the memory and the display device.

[Item 10] The drug injection device according to item 9, wherein:
the RF tag stores drug information including information indicating at least a type of the drug;
with the drug cartridge loaded in the housing space, the control device is configured to:
cause the transmission/reception circuit to transmit a control signal via the antenna;
cause the transmission/reception circuit to receive, via the antenna, first temperature information and drug information output from the first temperature sensor and the RF tag of the drug cartridge;
determine one set of control parameters from the control information stored in the memory based on the first temperature information and the drug information; and
control the motor using the determined set of control parameters.

[Item 11] The drug injection device according to item 10, wherein the motor driver generates a drive signal by pulse width modulation and outputs the drive signal to the motor.

[Item 12] The drug injection device according to item 11, wherein in the control information of each drug, a duty ratio of the drive signal by the pulse width modulation of a set of control parameters for the motor is smaller as a lower limit temperature of a temperature range of the set of control parameters for the motor is higher.

[Item 13] The drug injection device according to item 11 or 12, wherein:
in the control information of each drug, the set of control parameters of the motor for each temperature range includes an initial value and an increment of duty ratio of the drive signal by pulse width modulation; and
the control device causes the motor driver to generate the drive signal so that current flowing in the motor increase in steps.

[Item 14] The drug injection device according to any one of items 10 to 13, wherein the control device does not drive the motor when the first temperature information is in a first temperature range that is less than a first temperature or equal to or greater than a second temperature.

[Item 15] The drug injection device according to item 14, wherein when the first temperature information is in a second temperature range that is equal to or greater than a first temperature and less than a third temperature lower than the second temperature, the control device controls the display device to display information indicating to wait before injection.

[Item 16] The drug injection device according to item 15, wherein when the first temperature information is in a third temperature range that is equal to or greater than the third temperature and less than the second temperature, the control device controls the display device to display information indicating that it is possible to perform injection.

[Item 17] The drug injection device according to item 15, further including a second temperature sensor provided in the device housing and configured to output second temperature information indicating a temperature inside the device housing.

[Item 18] The drug injection device according to item 17, wherein when the second temperature information is equal to or greater than a predetermined temperature, the control device successively obtains the first temperature information at predetermined time intervals, successively calculates estimated time until it is possible to perform injection based on the first temperature information and the second temperature information, and controls the display device to display information indicating the calculated estimated time.

[Item 19] The drug injection device according to any one of items 8 to 18, wherein:
the antenna includes a first portion and a second portion, which can independently receive signals from outside; and
the first portion and the second portion are arranged orthogonal to each other adjacent to the housing space.

[Item 20] The drug injection device according to any one of items 8 to 19, further including:
a user interface including an inject button and configured to accept an instruction from an operator; and
a touch sensor of a surface charge transfer type, wherein:
the device housing has a skin-contact surface that comes into contact with skin of the operator during injection; and
the touch sensor is arranged on the skin-contact surface.

[Item 21] The drug injection device according to item 20, wherein:
when a signal resulting from pressing the inject button is received while a detection signal resulting from skin contact is received from the touch sensor, the control device controls the motor driver so as to perform an injection operation; and
when the touch sensor detects separation from skin during the injection operation, the control device causes the display device to display information indicating abnormality.

[Item 22] The drug injection device according to any one of items 8 to 21, further including:
a sensor having a first axis, a second axis and a third axis, which are orthogonal to each other, configured to detect acceleration along the axes or angular acceleration around the axes, and arranged in a housing so that one of the first axis, the second axis and the third axis coincides with a direction of movement of the piston; and
the control device causes the display device to display information regarding an attitude of the drug injection device based on a detection signal of the sensor.

[Item 23] The drug injection device according to any one of items 8 to 22, further including:
a rotary encoder including an encoder plate attached to a rotation axis of the motor and a pulse encoder, wherein:
the encoder plate includes one reference blade section and a plurality of normal blade sections arranged along a circumference, each including a notch and a blade;
the blades of the plurality of normal blade sections have an equal length in a circumferential direction, and the notches of the plurality of normal blade sections have an equal length in the circumferential direction;
a length of the blade of the reference blade section in the circumferential direction and a length of the notch in the circumferential direction are different respectively from the length of the blades of the plurality of normal blade sections in the circumferential direction and the length of the notches in the circumferential direction; and
the pulse encoder includes a light-emitting element and a light-receiving element arranged so as to receive light emitted from the light-emitting element, wherein the reference blade section and the plurality of normal blade sections of the encoder plate, which rotates in sync with the rotation of the motor, crosses an optical path between the light-emitting element and the light-receiving element, thereby generating a pulse signal including a pulse corresponding to the blade of the reference blade section and pulses corresponding to the blades of the plurality of normal blade sections.

[Item 24] The drug injection device according to item 23, wherein the length of the blade of the reference blade section in the circumferential direction is larger than the length of the blades of the normal blade sections in the circumferential direction, and the length of the notch of the reference blade section in the circumferential direction is smaller than the length of the notches of the normal blade sections in the circumferential direction.

[Item 25] The drug injection device according to item 23 or 24, wherein the control device controls the motor driver based on the pulse signal.

[Item 26] The drug injection device according to any one of items 23 to 25, wherein the control device calculates the number of blades of the reference blade section and the plurality of normal blade sections per one revolution of the encoder plate based on the pulse signal, and causes the display device to display information indicating a fault if the calculation result is different from a predetermined value.

[Item 27] The drug injection device according to item 22, wherein the control device reverses an orientation of information displayed on the display device based on a detection signal from the sensor.

[Item 28] The drug injection device according to any one of items 8 to 27, wherein the control device causes the display device to display the drug information.

[Item 29] The drug injection device according to any one of items 8 to 28, wherein the control device displays a theme color corresponding to a type of the drug across a plurality of operation screens.

[Item 30] The drug injection device according to item 8 or 9, wherein:
the device housing has a distal end section including a protruding portion extending in a longitudinal direction, a skin-contact surface located on an upper surface of the protruding portion, a device recess portion adjacent to the skin-contact surface, the housing opening being located at a bottom of the device recess portion, and an injection needle attachment portion that covers the housing opening and has an inner space in which a distal end of the drug cartridge is inserted;
the housing space is adapted to house at least a portion of the drug cartridge that is not housed in the cassette;
the drug injection device further including a hood including a hood body located in the device recess portion and pivotally attached to the device housing, a needle covering supported to be movable relative to the hood body, and a biasing member configured to bias the needle covering in a direction of the projecting position; and
the hood is pivotable between a first position at which the injection needle attachment portion is covered and a second position at which the injection needle attachment portion is exposed.

[Item 31] A drug injection system, including:
the cassette according to any one of items 4 to 7; and
the drug injection device according to any one of items 8 to 29.

[Item 32] The drug injection system according to item 31, further including the drug cartridge according to any one of items 1 to 3, wherein: the drug cartridge is housed in the cassette columnar space of the cassette.

[Item 33] A drug injection system, including:
the drug cartridge according to any one of items 1 to 3;
a cassette including a cassette columnar space in which at least a portion of the drug cartridge is housed; and
the drug injection device according to any one of items 8 to 29.

[Item 34] The drug injection system according to item 31 or 32, wherein the drug injection device includes:
a secondary battery; and
a charging terminal provided in the device housing, wherein:
   the device housing has a distal end section including a protruding portion extending in a longitudinal direction, a skin-contact surface located on an upper surface of the protruding portion, a device recess portion adjacent to the skin-contact surface, the housing opening being located at a bottom of the device recess portion; and
   with the cassette loaded in a housing space of the drug injection device, a portion of the hood of the cassette is exposed to an outside in the recess portion of the device housing, and another portion of the hood is located in the device housing so that the hood cannot pivot.

[Item 35] The drug injection system according to item 34, further including a charger including a power supply circuit for charging the secondary battery of the drug injection device, and a charger housing that houses the power supply circuit, wherein:
the charger housing includes a charger recess portion having a space in which the distal end portion of the drug injection device can be inserted, a step provided at a bottom of the charger recess portion and having a shape corresponding to the housing recess portion of the drug injection device, and a supply terminal located in the recess portion and connected to the power supply circuit; and
the charger housing is configured so that with the cassette not loaded in the drug injection device, the distal end section of the drug injection device can be housed in the charger recess portion so that the charging terminal of the drug injection device is in contact with the supply terminal, and with the cassette loaded in the drug injection device, the cassette interferes with the step of the charger housing, thereby preventing the distal end section of the drug injection device from being housed in the charger recess portion.

### First Embodiment

### (Outline of Drug Injection System)

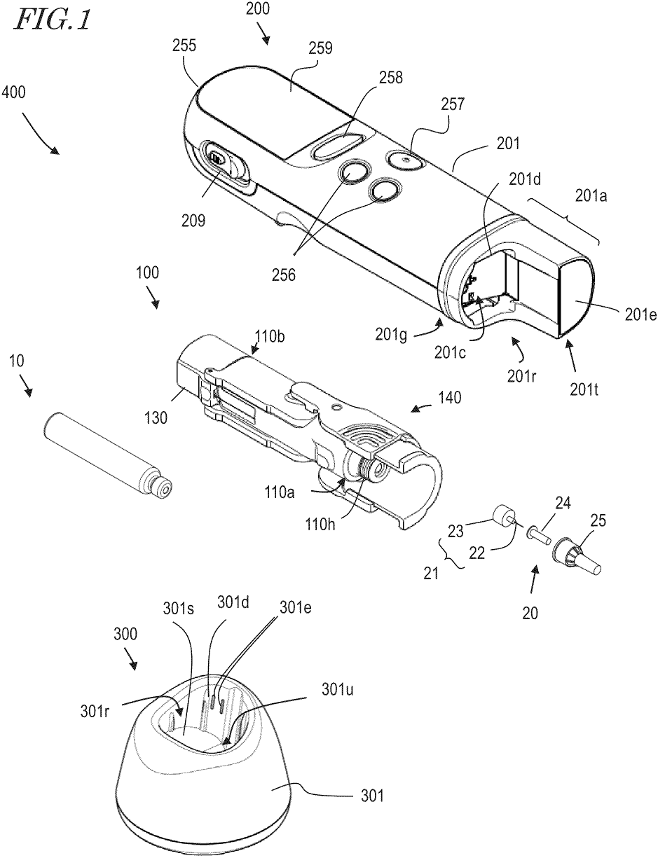

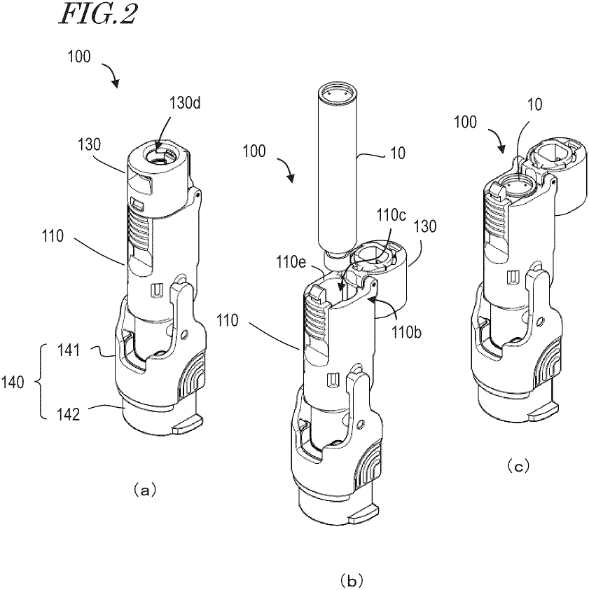

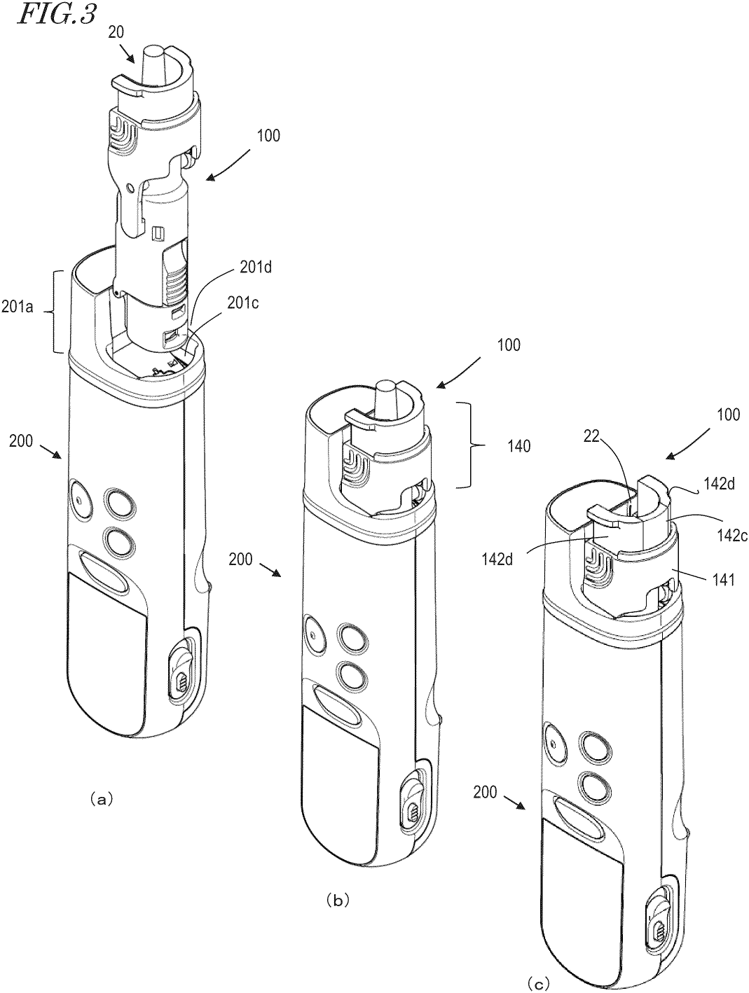

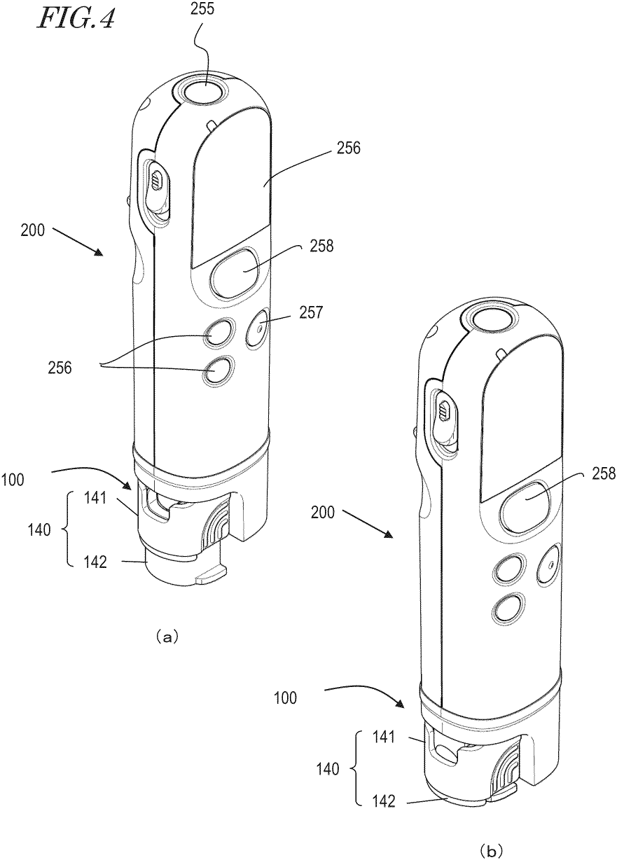

FIG. 1 is a perspective view showing a drug injection system 400 including a cassette 100, a drug injection device 200 and a charger 300. The cassette 100 houses the drug cartridge 10. FIG. 2(a) to FIG. 2(c) are perspective views illustrating how the drug cartridge 10 is housed in the cassette 100, and FIG. 3(a) to FIG. 3(c) are perspective views illustrating how the cassette 100 is housed in the drug injection device 200. FIG. 4(a) and FIG. 4(b) are perspective views illustrating the state where injection is performed. FIG. 5 is a block diagram illustrating an example configuration of an electrical circuitry of the drug injection device 200. With reference to these figures, the drug injection system will be outlined. Note that the drug injection system of the present disclosure is typically used by patients to perform injection by themselves. However, if a patient is young and it is not appropriate for the patient to handle the drug injection system himself/herself, a person other than the patient, such as a guardian, may handle the drug injection system.

The drug cartridge 10 holds multiple doses of drug to be injected into an operator such a as a patient, for example. As shown in FIG. 2(a), the cassette 100 includes a cassette body 110, a cassette cap 130 and a hood 140.

The cassette body 110 has a cassette columnar space 110c capable of housing at least a portion of the drug cartridge 10, an injection needle attachment portion 110h that is located at the distal end 110a of the cassette columnar space 110c and to/from which an injection needle can be attached/detached, and a body opening 110e that is located at the rear end 110b of the columnar space and accessible to the columnar space. The cassette cap 130 is supported in the vicinity of the rear end 110b of the cassette body 110 so that the cassette cap 130 can open and close the body opening 110e. The hood 140 includes a hood body 141 and a needle covering 142. The needle covering 142 is supported on the hood body 141 so that the needle covering 142 can move between a projecting position in which the needle covering 142 projects relative to the hood body 141 and a stowed position in which the needle covering 142 is at least partially stowed in the hood body 141.

As shown in FIG. 2(b), the cassette cap 130 is opened, the drug cartridge 10 is inserted into the cassette columnar space 110c and the cassette cap 130 is closed, thereby completing the loading of the drug cartridge 10 in the cassette 100. After starting to use a new drug cartridge 10, if the drug is still in the drug cartridge 10, the cassette 100 can be stored in a case (not shown), for example, and stored in a cold place, such as a refrigerator, with the drug cartridge 10 loaded in the cassette 100.

As will be described in detail below, in the present embodiment, the drug cartridge 10 includes a first temperature sensor and an RF tag. The first temperature information detected by the first temperature sensor 15 is transmitted by the RF tag to the drug injection device 200.

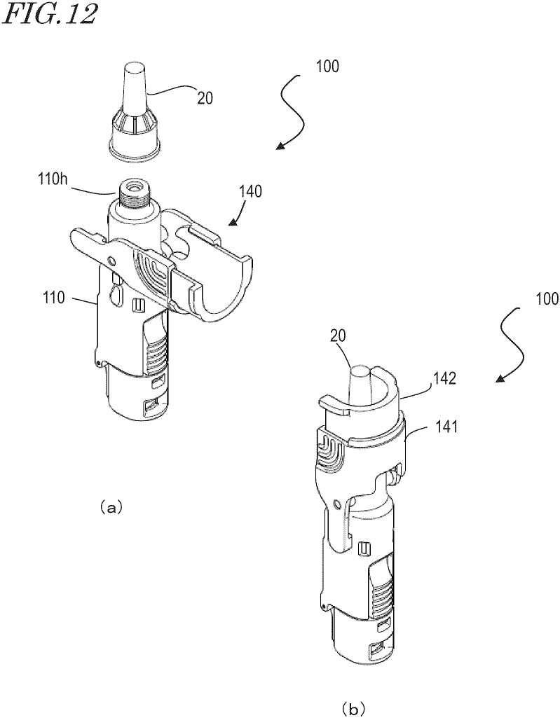

As shown in FIG. 12, an injection needle 21 can be attached to and detached from the injection needle attachment portion 110h of the cassette 100. The injection needle 21 is disposable, and is handled separately from the cassette 100 except when in use as a needle unit 20, for example. The needle unit 20 includes the injection needle 21, a needle cap 24 and a needle case 25. The injection needle 21 includes a needle 22 and a connecting portion 23 that supports the needle 22 and is detachably attached to the injection needle attachment portion 110h of the cassette 100. For example, a male thread may be provided on the distal end of the injection needle attachment portion 110h and a female thread may be provided on a connecting portion 23 of the injection needle 21. The needle cap 24 has a cylindrical shape that covers the needle 22, and the needle case 25 houses the injection needle 21 with the needle 22 covered by the needle cap 24.

Note that, as shown in FIG. 1, the drug cartridge 10, the cassette 100 and the drug injection device 200 each have the longitudinal direction L. In the present application, one of the opposite ends of the drug cartridge 10, the cassette 100 and the drug injection device 200 in the longitudinal direction to which the injection needle 21 is attached is referred to as the distal end, the distal end section or the distal end portion. The end opposite to the distal end in the longitudinal direction of the drug cartridge 10, the cassette 100 and the drug injection device 200 is referred to as the rear end, the rear end section or the rear end portion.

The drug injection device 200 has a device housing 201. The device housing 201 has a cylindrical shape having such a thickness that is easy for the operator to hold with one hand, for example. In the present embodiment, the device housing 201 has an oblong circle shape in a cross section perpendicular to the longitudinal direction, making it easy for the operator to hold. However, the shape of the device housing 201 is not limited to this and may be a cylindrical shape or a rectangular cylindrical shape.

A distal end section 201a, which is one of the opposite ends of the device housing 201 in the longitudinal direction, has a protruding portion 201t extending in the longitudinal direction and a device recess portion 201r. The device recess portion 201r is adjacent to the protruding portion 201t, and the protruding portion 201t and the device recess portion 201r are formed by providing a gap in the cylindrical shape of the device housing 201 that cuts out a portion of the end surface and a portion of the side surface of the cylindrical shape. The upper surface of the protruding portion 201t is referred to as the skin-contact surface 201e. A housing opening 201d into which the cassette 100 can be inserted is located at the bottom surface of the device recess portion 201r. The drug injection device 200 has a housing space 201c within the device housing 201 capable of housing at least a portion of the cassette 100, and the housing space 201c is connected to the housing opening 201d.

The drug injection device 200 has a power button 255, a select button 256, an OK button 257, an inject button 258, an eject lever 209 and a display device 259 on the surface of the device housing 201. The surface on which these buttons and the display device 259 are located is called the front surface. The power button 255, the select button 256, the OK button 257 and the inject button 258 are examples of user interfaces that receive instructions from the operator. Some or all of the user interfaces may be a touch panel provided on the display device 259. A charging terminal 201 g is arranged on the distal end section 201a of the device housing 201, which will be described below.

When the drug injection system 400 is used, when the power button 255 is pressed to start the drug injection device 200, the display device 259 displays the operating procedure of the drug injection device 200, and the drug information, the injection history, etc., of the drug cartridge 10 in the loaded cassette 100.

As shown in FIG. 3(a) to FIG. 3(c), the cassette 100, to which the needle unit 20 is attached, is loaded in the drug injection device 200, and the needle case 25 and the needle cap 24 are removed. In this state, the needle 22 is located within the space enclosed by the needle covering 142, and the distal end of the needle 22 does not project from the needle covering 142.

After pressing the select button 256 and the OK button 257 as appropriate to determine the operation of the drug injection device 200, a drug injection operation is performed. The drug injection device 200 of the present embodiment is of a semi-automatic type, with needle insertion and needle removal being manual, i.e., performed by the operator. As shown in FIG. 4(a) and FIG. 4(b), in the state where the drug injection device 200 is held so that the distal end of the needle covering 142 is in contact with the skin, when the drug injection device 200 is pressed against the skin, the needle covering 142 retracts into the hood body 141. Accordingly, the distal end of the needle 22 comes into contact with the skin, and the injection needle 22 is inserted into the skin to a predetermined depth. Then, when the inject button 258 is pressed, a predetermined amount of drug is injected from the drug cartridge 10.

After completion of the drug injection, as the operator moves the drug injection device 200 off the skin, the injection needle 21 is pulled out of the skin. Then, the eject lever 209 is operated to eject the cassette 100 from the drug injection device 200.

As shown in FIG. 5, the drug injection device 200 includes a control section 251 including an arithmetic unit such as a CPU, a secondary battery 253 as a power source, a charging section 252 including a charging circuit for charging the secondary battery 253, a memory 254 storing computer programs, data, etc., and a clock 261. The control section 251 and the memory 254 together form a control device 280, and the control section 251 reads a program stored in the memory 254 and controls various components shown in FIG. 5 according to the procedure of the computer program. The procedure of the computer program will be shown by the following description and by the flow charts of the accompanying drawings. The drug injection device 200 may further include a buzzer 260 that informs the operator by means of sound.

The drug injection device 200 further includes a motor driver 263, a motor 264 and a rotary encoder 265. The motor driver 263, the motor 264 and the rotary encoder 265 together form a portion of the piston drive mechanism as will be described below.

The drug injection device 200 also includes various detectors to detect the status of various parts of the drug injection device 200. Specifically, the drug injection device 200 includes a piston origin detector 271, a cassette loading detector 272, an eject lever detector 274, a touch sensor 275 and an acceleration sensor 276. The drug injection device 200 may further include a second temperature sensor 273.

The drug injection device 200 includes an RF-ID reader 277. The drug injection device 200 may further include an RF-ID writer. The RF-ID reader 277 reads the first temperature information of the first temperature sensor 15 transmitted from an RF tag 16 of the drug cartridge 10, and drug information including information indicating the type of the drug stored in the memory of the RF tag 16. The read information is input to the control section 251, which uses the obtained first temperature information to control the motor and control the operation of the drug injection device 200.

The drug injection device 200 may further include a communication section 262. The communication section 262 transmits and receives information to and from the outside by, for example, infrared communication, wireless communication, etc. Specifically, the communication section 262 may be a transmitter/receiver that uses a short-range wireless communication standard, such as BLE (Bluetooth Low Energy, Bluetooth is a registered trademark). For example, the time when the operator uses the drug injection device 200, the type of drug, the amount of drug injected, and other information may be stored in the memory 254 at the time of use, and at a predetermined timing, these information may be transmitted using the communication section 262 to a portable device such as a smartphone, a tablet device, or to an external device such as a dedicated device for managing the drug injection device 200. The information may be transmitted from a portable device to a server, or the like, of a hospital, a drug manufacturer, etc., via a cellular phone line or an Internet line.

The charger 300 includes a charger housing 301 and a power supply circuit arranged in the charger housing 301. The charger housing 301 includes a charger recess portion 301r with a space into which the distal end section 201a of the drug injection device 200 can be inserted.

The charger housing 301 includes a step 301s provided at the bottom of the charger recess portion 301r and having a shape corresponding to the device recess portion 201r provided in the distal end section 201a of the drug injection device 200, a space 301u within the charger recess portion 301r and adjacent to the step 301s, and a supply terminal 301e located in the charger recess portion 301r and connected to the charging circuit. A plurality of ribs 301d extending in the depth direction of the charger recess portion 301r are provided on the side surface of the charger recess portion 301r.

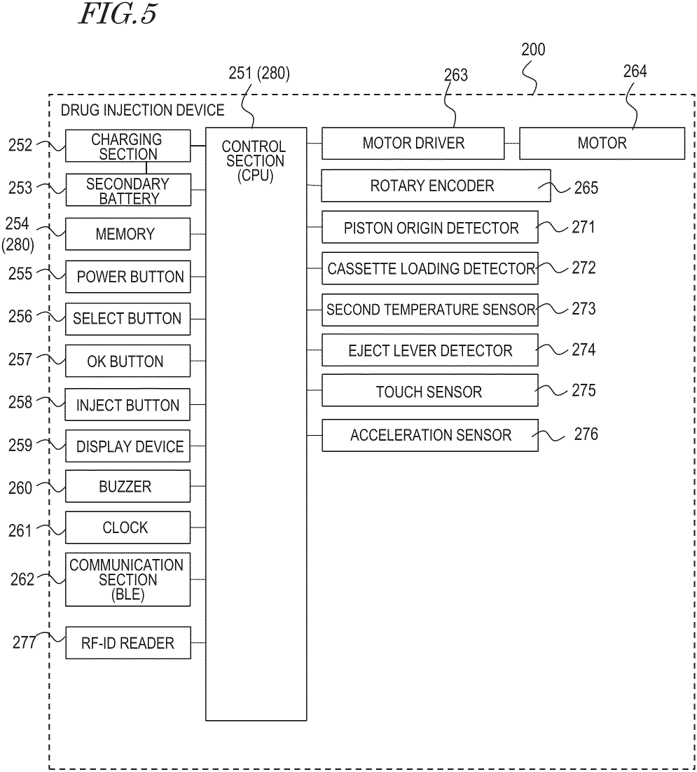

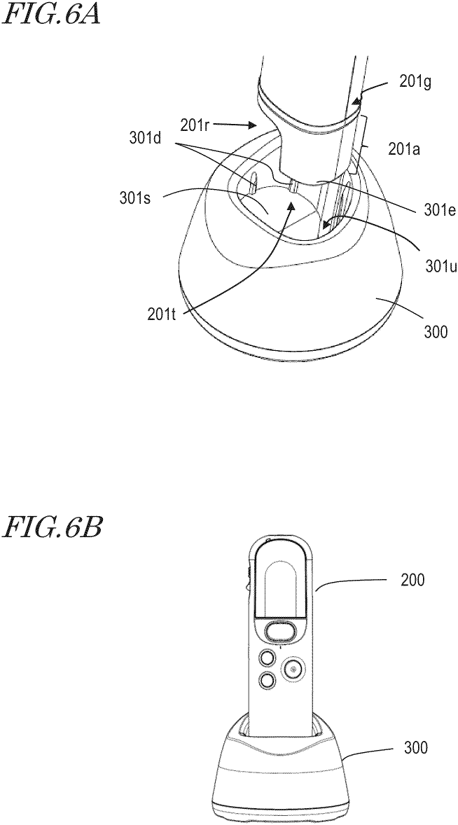

FIG. 6A is a view illustrating how the drug injection device 200 is set in the charger 300, and FIG. 6B is a front view showing the drug injection device 200 set in the charger 300. In order to charge the drug injection device 200 with the charger 300, the cassette 100 is removed from the drug injection device 200, and the distal end section 201a of the drug injection device 200 is inserted into the charger recess portion 301r of the charger 300. At this time, since the device recess portion 201r of the drug injection device 200 corresponds to the step 301s, the protruding portion 201t is inserted into the space 301u beside the step 301s without interference, and the entire distal end section 201a can be inserted into the charger recess portion 301r, as shown in FIG. 6B. This allows the distal end section 201a of the drug injection device 200 to be housed in the charger recess portion 301r so that the charging terminal 201g of the drug injection device 200 is in contact with the supply terminal 301e. At this time, the side surface of the distal end section 201a of the drug injection device 200 is in contact with the ribs 301d of the charger 300. Therefore, a space is formed between the side surface of the charger recess portion 301r and the side surface of the distal end section 201a of the drug injection device 200. This space allows the heat of the secondary battery generated by charging to be released to the outside of the charger recess portion 301r.

As shown in FIG. 6B, the charger 300 can hold the drug injection device 200 so that the drug injection device 200 is in the upright position. This allows the charger 300 to function as a storage place for the drug injection device 200 when not in use, and to store the drug injection device 200 in a space-saving and more conspicuous manner than when the drug injection device 200 is stored on its side or the drug injection device 200 is housed and charged inside a case.

On the other hand, when the cassette 100 is loaded in the drug injection device 200, a portion of the cassette 100 protrudes into the device recess portion 201r. Therefore, even if one tries to insert the distal end portion 201a of the drug injection device 200 into the charger recess portion 301r of the charger 300, the cassette 100 interferes with the step 301s of the charger recess portion 301r, thereby preventing the protruding portion 201t from being inserted into the space 301u beside the step 301s, and the entire distal end section 201a from being inserted into the charger recess portion 301r. Therefore, the distal end section 201a of the drug injection device 200 cannot be housed in the charger recess portion 301r, and the drug injection device 200 cannot be set in the charger 300.

Thus, with the drug injection system 400 of the present embodiment, the drug injection device 200 cannot be loaded in the charger 300 with the cassette 100 inserted. This ensures that the cassette 100 be removed during charging, thereby preventing the drug in the cassette 100 from deteriorating due to the heat generated by the drug injection device 200 during charging.

With the drug injection system of the present embodiment, the drug cartridge 10 includes the first temperature sensor 15, thereby allowing the temperature of the drug stored in the drug cartridge 10 to be measured. This information can be used to estimate the viscosity of the drug so as to drive the motor with a drive force in accordance with the viscosity, and determine whether the temperature is suitable for injection so as to control the operation of the drug injection device 200. The drug cartridge 10, the cassette 100 and the drug injection device 200 will now be described in detail.

### Drug Cartridge 10

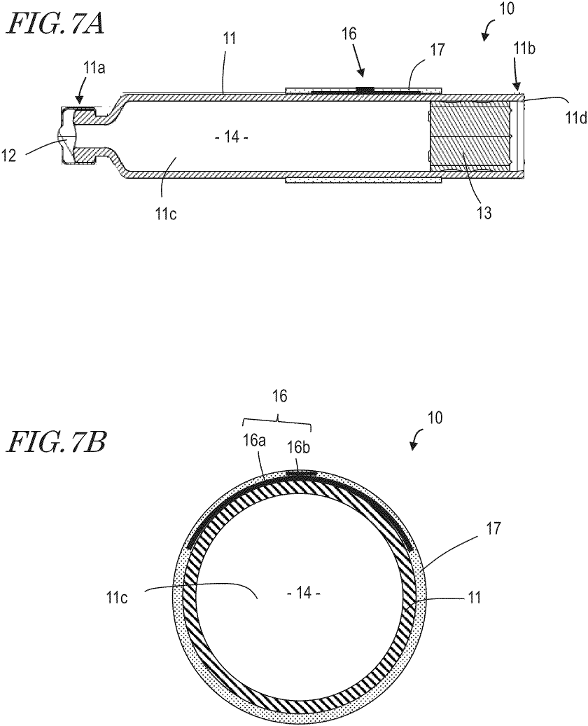

An example of the drug cartridge 10 of the present embodiment will be described. FIG. 7A is a cross section parallel to the longitudinal direction of the drug cartridge 10, and FIG. 7B is a cross section perpendicular to the longitudinal direction of the drug cartridge 10. The drug cartridge 10 includes a cylinder 11, a cylinder cap 12, a gasket 13, a drug 14 and an RF tag 16.

The cylinder 11 has a first end 11a and a second end 11b, which are spaced apart from each other in the longitudinal direction, and a cylinder columnar space 11c located between the first end 11a and the second end 11b. The needle 22 of the injection needle 21 can be inserted and pulled out through the first end 11a. For example, the cylinder 11 has an outer shape that tapers on the first end 11a side so that the cross section perpendicular to the longitudinal direction of the cylinder columnar space 11c decreases on the first end 11a side, and the opening of the cylinder columnar space 11c on the first end 11a side is sealed with a rubber cylinder cap 12. The cylinder 11 has a cylinder opening 11d connected to the cylinder columnar space 11c at the second end 11b.

The gasket 13 is inserted into the cylinder columnar space 11c through the cylinder opening 11d and is supported on the inner wall of the cylinder 11 so that the gasket 13 can move in the longitudinal direction.

The first end 11a side and the second end 11b side of the cylinder columnar space 11c are closed by the cylinder cap 12 and the gasket 13, and the drug 14 is sealed in the closed cylinder columnar space 11c. The drug 14 contains at least a liquid first component and is liquid at room temperature.

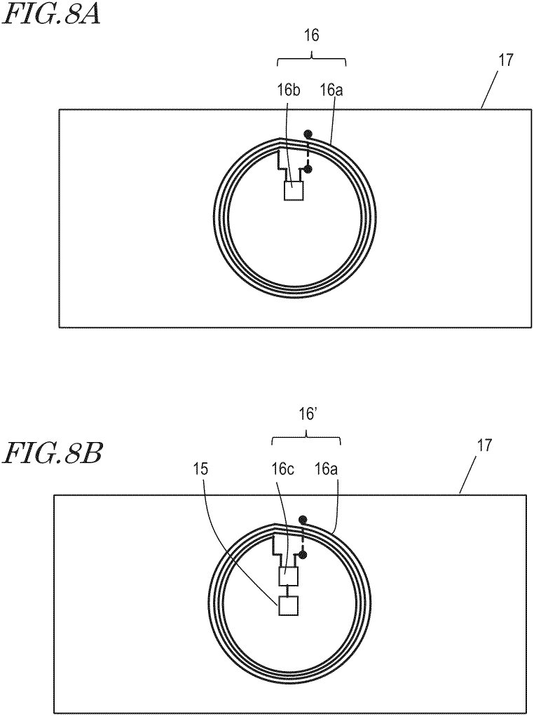

FIG. 8A and FIG. 8B are schematic plan views of the RF tag 16. The RF tag 16 is a device that stores identification information of an object to which the tag is attached, and transmits the identification information wirelessly. In the present embodiment, the RF tag 16 stores drug information including at least information indicating the type of drug in the cylinder columnar space 11c, and wirelessly transmits the information indicating the type of drug and the first temperature information indicating the temperature detected by the first temperature sensor to the outside in response to an instruction from the outside.

The RF tag 16 may be of an active type or a passive type. In the present embodiment, the RF tag 16 is of a passive type and is an RF tag with a temperature sensor. For example, RF tag 16 includes an antenna 16a and an IC 16b. The antenna 16a transmits and receives electromagnetic waves in the long wave, short wave, or microwave bands. The IC 16b includes a transmitter section, a receiver section, a storage section and a power rectifier section. The IC 16b of the present embodiment further includes a first temperature sensor.

As shown in FIG. 8B, an RF tag 16′ may include an antenna 16a and an IC 16c without a temperature sensor. In this case, the drug cartridge 10 further includes a first temperature sensor 15 electrically connected to the IC 16c. The RF tag 16 or the RF tag 16′ and the first temperature sensor 15 are supported on a label 17 by attaching or laminating, for example. The label 17 is attached to the outer surface of the cylinder 11. On the outside of the attached label 17, the name of the drug 14, or the like, may be written. The drug information to be described below may be written in letters, pictograms, etc.

The first temperature sensor detects the temperature around the second temperature sensor. In the present embodiment, the IC 16b including the first temperature sensor is attached to the cylinder 11 together with the label, and thus directly detects the temperature of the cylinder 11. The temperature of the cylinder 11 is the same as the temperature of the drug in the cylinder columnar space 11c. Therefore, it can be said that the first temperature sensor detects the temperature of the drug in the drug cartridge 10.

The storage section stores drug information about the drug in the cylinder columnar space 11c. The drug information includes at least information indicating the type of drug. The drug information may further store information indicating the expiration date of the drug, information indicating the initial amount of drug in an unused state, information about the manufacture of the drug such as the manufacturing lot, unique identification information of the drug cartridge 10, information about the viscosity of the drug, etc.

With the RF tag 16, when the antenna 16a receives a signal transmitted from the drug injection device 200 described below, electromotive force due to resonance is generated, and the power rectifier of the IC 16b rectifies the electromotive force, thereby generating electric power to drive the RF tag 16. This activates the IC 16b, which reads out the drug information stored in the storage section and the first temperature information indicating the temperature detected by the first temperature sensor, and the transmitter section converts the read-out information into electromagnetic waves and transmits the information to the outside through the antenna 16a. The transmitted information is received by the drug injection device and used to control the drug injection device, as will be described below.

The drug cartridge 10, which includes the first temperature senso, can detect the temperature of the drug upon request from the drug injection device 200. Thus, it is possible to realize a drug injection device such that when a cold-stored drug cartridge is used for injection, the temperature of the drug can be used to determine whether the temperature of the drug is suitable for injection, to estimate the viscosity of the drug according to the temperature of the drug, and to inject the drug with an appropriate drive force.

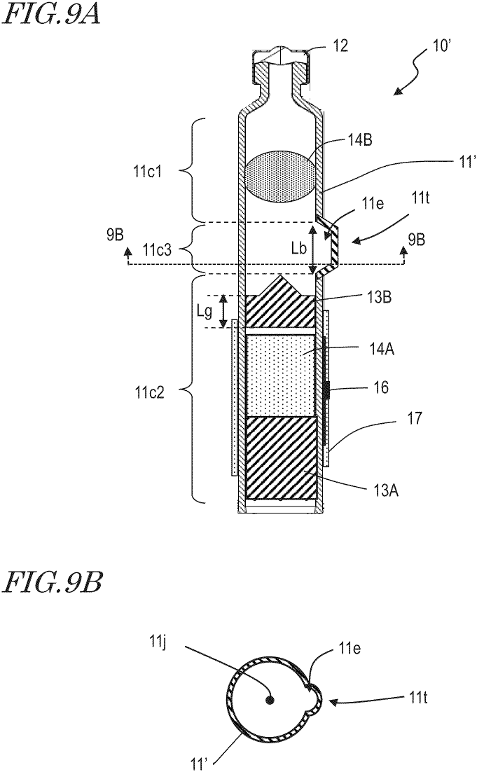

Although the drug cartridge 10 contains only a liquid component, the drug cartridge of the present disclosure may contain a solid component and a liquid component. FIG. 9A shows a schematic cross section of a drug cartridge 10′ containing a drug in which a liquid component and a solid component are held separate in an unused state, and FIG. 9B shows a cross section taken along line 9B-9B of FIG. 9A.

The drug cartridge 10′ includes a cylinder 11′, a first gasket 13A, a second gasket 13B, and a liquid component 14A containing a first component of the drug 14 and a solid component 14B containing a second component thereof. The liquid component 14A is liquid at room temperature and the solid component 14B is solid at room temperature. The solid component 14B is, for example, in contact with and supported by the inner side surface of the cylinder 11′. The label 17 and the RF tag 16 are arranged on the side surface of the cylinder 11′.

The cylinder 11′ has a cylinder columnar space 11c, and the cylinder columnar space 11c includes a first region 11c1 located on the first end 11a side, a second region 1102 located on the second end 11b side, and a third region 11c3 sandwiched between the first region 11c1 and the second region 11c2 .

The side surface of the cylinder 11′ includes a projecting portion 11t projecting outwardly with respect to the axis 11j in the third region 11c3. The projecting portion 11t extends in the longitudinal direction and defines a bypass space 11e arranged adjacent to the cylinder columnar space 11c in a cross section perpendicular to the longitudinal direction. The length Lb of the bypass space 11e in the longitudinal direction is longer than the length Lg over which the second gasket 13B is in contact with the inner side surface of the cylinder 11′ (Lb>Lg).

In the initial state where the drug cartridge 10′ is unused, at least a portion of the second gasket 13B is located in the second region 11c2. The first gasket 13A is located on the second end 11b side of the second region 1102 in the cylinder columnar space 11c. The liquid component 14A of the drug is located in the second region 1102 and sandwiched between the first gasket 13A and the second gasket 13B. On the other hand, the solid component 14B is located in the first region 11c1 of the cylinder columnar space 11c.

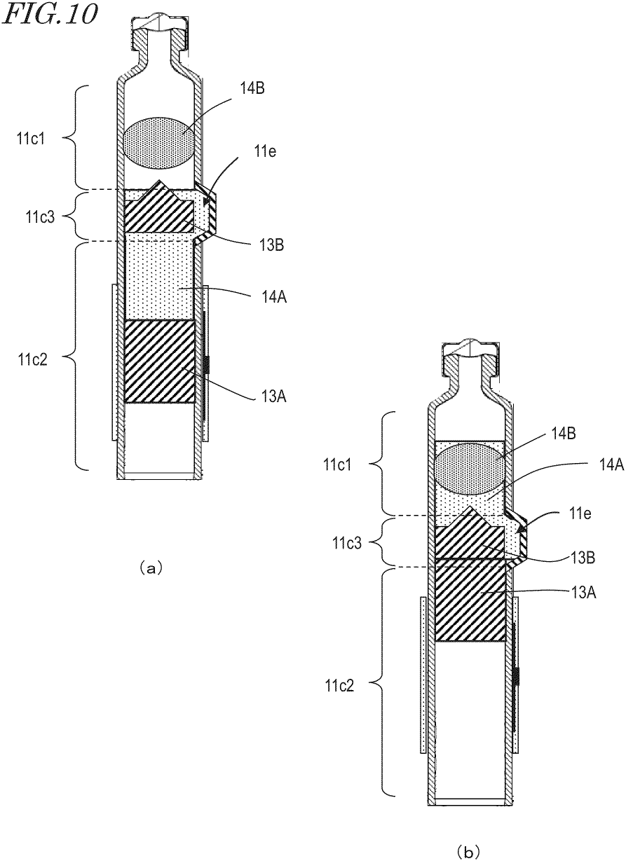

In the drug cartridge 10′, the solid component 14B is dissolved in the liquid component 14A before use. FIG. 10(a) and FIG. 10(b) are schematic diagrams illustrating the movement of the liquid component 14A while dissolving the solid component 14B. When the cassette 100 with the drug cartridge 10′ inserted therein is loaded in the drug injection device 200, a piston 210 of the drug injection device 200 moves the first gasket 13A forward. While the rear end of the second gasket 13B is in the second region 11c2, the space between the first gasket 13A and the second gasket 13B is sealed, so that as the first gasket 13A moves forward, the second gasket 13B and the liquid component 14A moves forward together.

As shown in FIG. 10(a), when the rear end of the second gasket 13B reaches the third region 11c3, the first region 11c1 located forward of the second gasket 13B and the second region 1102 located rearward of the second gasket 13B are connected together by the bypass space 11e, because the length Lg of the second gasket 13B in the longitudinal direction is shorter than the length Lb of the bypass space 11e in the longitudinal direction. This allows the liquid component 14A to flow through the bypass space 11e into the first region 11c1. In the meantime, the second gasket 13B does not move even when the first gasket 13A moves forward, and only the liquid component 14A moves into the first region 11c1. Thus, the liquid component 14A comes into contact with the solid component 14B, and the solid component 14B dissolves into the liquid component 14A.

As shown in FIG. 10(b), when all the liquid component 14A moves to the first region 11c1, the first gasket 13A contacts the second gasket 13B. Thus, the first gasket 13A and the second gasket 13B thereafter move forward together while being in contact with each other.

As will be described below, the solid component 14B in contact with the liquid component 14A dissolves into the liquid component 14A as the entire drug cartridge 10′ is oscillated by the operator.

Thus, even if the drug held in the drug cartridge contains a solid component and a liquid component, the drug information can be stored in the storage section of the RF tag 16. Thus, as the stored drug information and the first temperature information of the drug are transmitted to the drug injection device 200, it is possible to control the drug injection device 200 by a different procedure than a drug cartridge containing only a liquid component, or to perform a dissolution operation depending on the temperature of the drug.

### Cassette 100

As described with reference to FIG. 2, the cassette 100 includes the cassette body 110, the cassette cap 130 and the hood 140. The cassette 100 of the present embodiment, having the hood 140, allows the operator to more safely handle the cassette 100 or the drug injection device 200 with the cassette 100 attached while the injection needle 21 is attached.

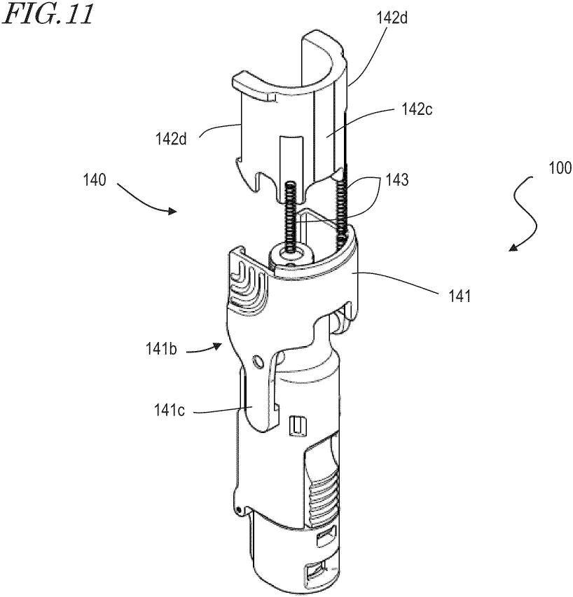

FIG. 11 is an exploded perspective view of the hood 140 of the cassette 100. The hood 140 includes the hood body 141, the needle covering 142 and a biasing member 143. The needle covering 142 has a generally U-letter shape in a cross section perpendicular to the longitudinal direction. Similarly, the hood body 141 also includes a portion having a generally U-letter shape in a cross section perpendicular to the longitudinal direction. The needle covering 142 is movably supported in the longitudinal direction with respect to the hood body 141. The biasing member 143 biases the needle covering 142 in the direction of projecting from the hood body 141. While the biasing member 143 is a spring in the present embodiment, it may be any other elastic member.

The needle covering 142 is at least partially transparent. In the present embodiment, the needle covering 142 includes a transparent first portion 142c and semi-transparent second portions 142d. More specifically, a first portion 142c is a region extending in the longitudinal direction, and is located at the bottom of the generally U-letter shape cross section in a cross section perpendicular to the longitudinal direction, for example. The second portions 142d are located so as to sandwich the first portion 142c.

The hood body 141 is pivotably supported by the cassette body 110 at the rear end 141b in the longitudinal direction. Thus, the hood 140 can pivot between the first position at which the injection needle attachment portion is covered and the second position at which the injection needle attachment portion is exposed. The hood body 141 has an arm 141c connected to the rear end 141b and extending toward the opposite side of the hood body 141 in the longitudinal direction. The hood body 141 is made of an opaque material, for example. Transparent and opaque may be colorless or colored.

FIG. 12(a) and FIG. 12(b) are views illustrating how the needle unit 20 is attached to the cassette 100. The drug cartridge 10 is inserted into the cassette 100 in advance. As shown in FIG. 12(a), the hood 140 is first rotated to the second position. In this state, the hood 140 does not cover the injection needle attachment portion 110h of the cassette 100, and the injection needle attachment portion 110h is exposed. Particularly, the hood 140 is located downward (on the rear end side) relative to the injection needle attachment portion 110h. Thus, for example, when the operator attaches the needle unit 20 to the injection needle attachment portion 110h, the operator’s hand does not come into contact with the hood 140, making the attachment easy. As shown in FIG. 12(b), after completion of the attachment of the needle unit 20, the hood 140 is rotated to the first position. Thus, a portion of the needle unit 20 excluding the distal end section 201a is covered by the hood 140 from three directions.

As shown in FIG. 3(a), the cassette 100 is inserted into the housing space 201c through the housing opening 201d of the drug injection device 200. FIG. 3(b) shows a state where the cassette 100 is completely loaded. In this state, a portion of the hood 140 is exposed in the device recess portion 201r of the distal end section 201a, and a portion is located within the housing opening 201d. Specifically, at least the arms 141c of the hood body 141 are located within the housing opening 201d. Therefore, in this state, for example, even if the operator attempts to rotate the hood 140 to the second position, i.e., to open the hood 140 so as to expose the needle unit 20, the arms 141c come into contact with the internal housing of the drug injection device 200 in the housing space 201c, and the hood 140 cannot be rotated. When the cassette 100 is loaded in the drug injection device 200, a portion of the hood body 141 encloses the device recess portion 201r of the distal end section 201a. This allows the drug injection device 200 to be held stable and pressed against the skin during administration.

As shown in FIG. 3(c), in this state, the operator pulls the needle case 25 of the needle unit 20 to remove the needle cap 24 and the needle case 25 from the needle unit 20, exposing the injection needle 21. The exposed injection needle 21 has its distal end located lower than the distal end of the needle covering 142.

The semi-transparent second portion 142d of the needle covering 142 faces the same direction as the front surface where the display device 259 of the drug injection device 200, etc., are arranged. Therefore, when the operator performs injection, the operator visually recognizes the injection needle 21 through the second portion 142d of the needle covering 142. With the second portion 142d being semi-transparent, the operator can recognize that the needle 22 is attached though the operator indistinctly recognizes the shape of the injection needle 21. Thus, the operator can confirm that the injection needle 21 is correctly attached, and it is possible to reduce the fear caused by not clearly recognizing the needle 22.

The first portion 142c of the needle covering 142 is transparent and faces the same direction as the side surface of the drug injection device 200. Thus, the operator can clearly visually recognize the injection needle 21 through the first portion 142c. As will be described below, for example, when performing an air-removing operation in the drug cartridge 10, it is possible to confirm through the first portion 142c that the drug is seeping out from the distal end of the injection needle 21 and to determine that the air removal is complete.

When performing injection, by pushing down the drug injection device 200 while having the distal end of the needle covering 142 against the skin, the needle covering 142 retracts and is stored in the hood body 141, causing the injection needle 21 to relatively project from the distal end of the needle covering 142. This allows the injection needle 21 to be inserted into the skin until the skin-contact surface 201e of the protruding portion 201t of the drug injection device 200 comes into contact with the skin.

When the injection is complete and the drug injection device 200 is removed from the skin, the needle covering 142 projects toward the distal end side by the biasing member 143, thereby covering the injection needle 21 again.

While the cassette 100 housing the drug cartridge 10 has been described above, the cassette of the present embodiment can be similarly configured also when housing the drug cartridge 10′ described above.

### Drug Injection Device 200

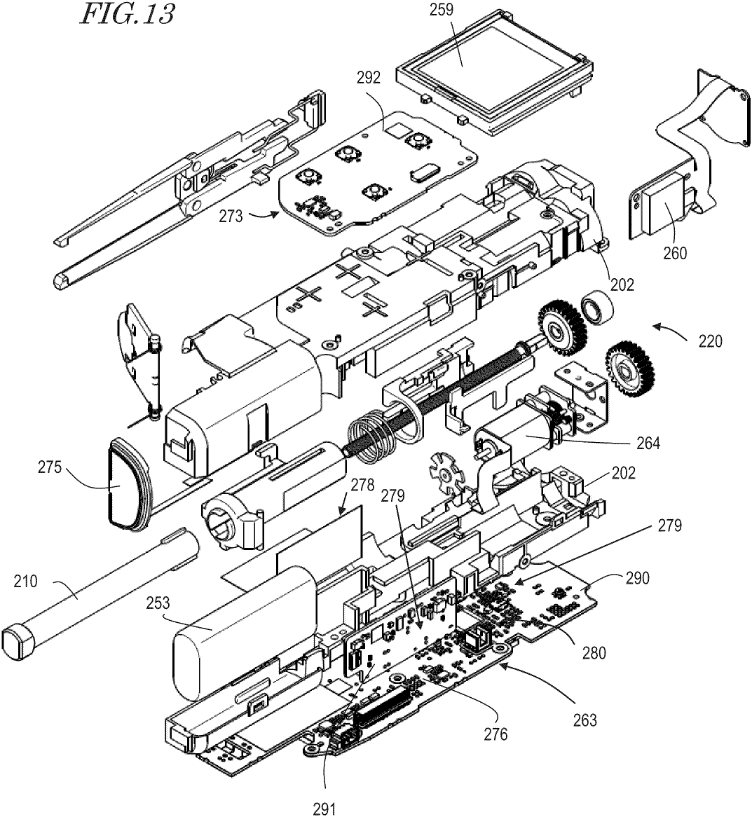

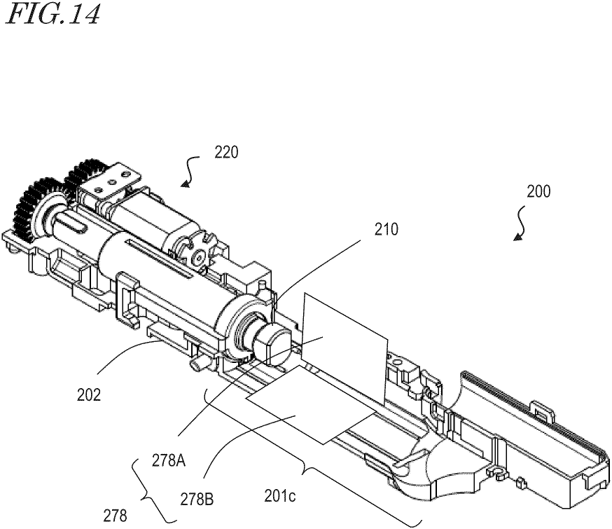

FIG. 13 is an exploded perspective view of the drug injection device 200 with the device housing 201 removed, and FIG. 14 is a perspective view of the drug injection device 200 showing the arrangement of the RF-ID antenna. FIG. 15 is an exploded perspective view of a piston drive mechanism 220 of the drug injection device 200. In addition to the device housing 201 and the control device 280 described above, the drug injection device 200 includes the piston 210 and the piston drive mechanism 220. In the present embodiment, the drug injection device 200 further includes an internal housing 202, a main board 290, a first sub-board 291 and a second sub-board 292. The internal housing 202 supports the piston drive mechanism 220. The control device 280 and the motor driver 263 are formed on the main board 290. The acceleration sensor 276 is also arranged on the main board 290.

The RF-ID reader 277 includes an antenna 278 and a transmission/reception circuit 279. The transmission/reception circuit 279 is formed, for example, on the main board 290. The select button 256, the OK button 257, the inject button 258 and the second temperature sensor 273 are arranged on the second sub-board 292. The second temperature sensor 273 is preferably arranged at a location where it is less susceptible to heat generated by various components in the device housing 201 during operation.

With the cassette 100, into which the drug cartridge 10 is inserted, loaded in the drug injection device 200, the RF-ID reader 277 reads the first temperature information transmitted from the drug cartridge 10 and the drug information including information that indicates the type of drug stored in the memory of the RF tag 16. The drug injection device 200 injects the drug from the drug cartridge 10 into the operator by moving the piston 210 based on these information. The structure of the RF-ID reader 277 and the piston drive mechanism 220 of the drug injection device 200 will be described primarily below.

### RF-ID Reader 277

As mentioned above, the RF-ID reader 277 includes the antenna 278 and the transmission/reception circuit 279. As shown in FIG. 14, the antenna 278 includes a first portion 278A and a second portion 278B, which can independently receive signals from the outside. The first portion 278A and the second portion 278B of the antenna 278 are arranged adjacent to the housing space 201c. The first portion 278A and the second portion 278B each include a generally planar radiating conductor and are preferably arranged orthogonal to each other.

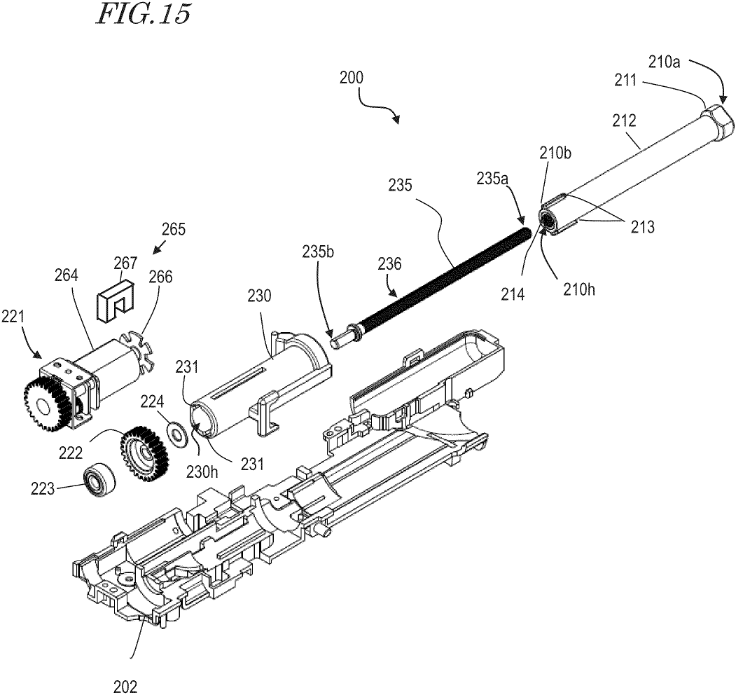

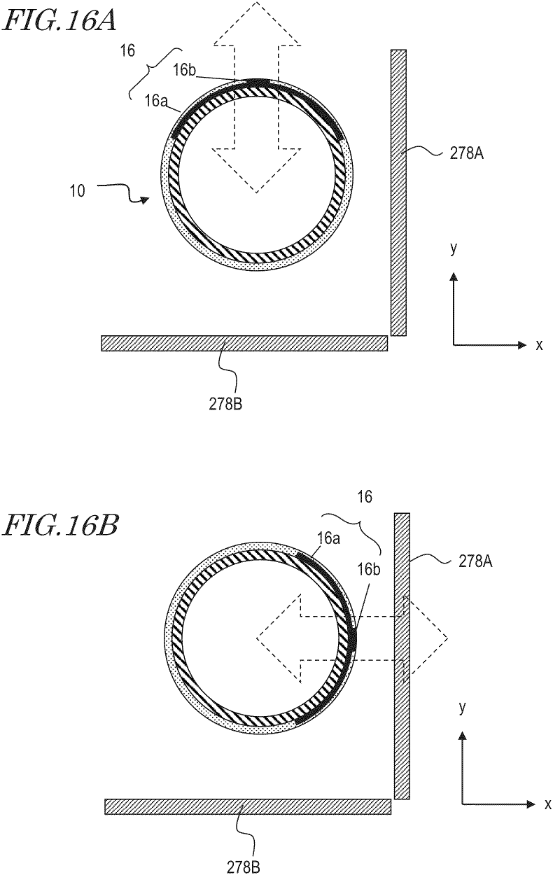

FIG. 16A and FIG. 16B schematically illustrate the positional relationship between the RF tag 16 and the first portion 278A and second portion 278B of the antenna 278 in the state where the cassette 100 into which the drug cartridge 10 is inserted is loaded in the drug injection device 200. In FIG. 16A and FIG. 16B, the cassette 100 is not shown. In the present embodiment, the drug cartridge 10 has a cylindrical shape, and the outer shape of the cross section perpendicular to the longitudinal direction has a generally circular shape. Therefore, it can be inserted in any orientation (angle) around its axis with respect to the cassette columnar space 110c of the cassette 100. Thus, when the cassette 100 is loaded in the drug injection device 200, the flat RF tag 16 can face any direction.

On the other hand, the antenna 16a of the RF tag 16 has a generally flat shape and has directionality. Specifically, the antenna 16a transmits electromagnetic waves with a large field strength distribution in a direction perpendicular to the planar shape. Therefore, if the antenna 278 of the RF-ID reader 277 has high receiving sensitivity only in one direction, the direction in which the receiving sensitivity of the antenna 278 is high and the direction in which the field strength of the electromagnetic wave transmitted from the antenna 16a of the RF tag 16 is strong are orthogonal or nearly orthogonal to each other, and the electromagnetic waves transmitted from the RF tag 16 may not be correctly received.

In the present embodiment, the first portion 278A and the second portion 278B of the antenna 278 are arranged orthogonal to each other, so that the antenna 278 has high sensitivity in the two orthogonal directions. Thus, for example, as shown in FIG. 16A, when the antenna 16a of the RF tag 16 is spread generally perpendicular to the y direction, the antenna 278 cannot receive electromagnetic waves from the antenna 16a of the RF tag 16 with strong sensitivity in the first portion 278A, but electromagnetic waves from the antenna 16a of the RF tag 16 can be received with sufficient sensitivity in the second portion 278B. As shown in FIG. 16B, when the antenna 16a of the RF tag 16 is spread generally perpendicular to the x direction, the antenna 278 cannot receive electromagnetic waves from the antenna 16a of the RF tag 16 with strong sensitivity in the second portion 278B, but electromagnetic waves from the antenna 16a of the RF tag 16 can be received with sufficient sensitivity in the first portion 278A. Even when the antenna 16a of the RF tag 16 is oriented in a direction other than those shown in FIG. 16a and FIG. 16b, the first portion 278A or the second portion 278B of the antenna 278 can receive the electromagnetic waves from the antenna 16a of the RF tag 16 with sufficient sensitivity. Thus, with the drug injection device 200 of the present embodiment, it is possible to more reliably read, from the drug cartridge 10 in the cassette 100, the first temperature information and drug information including the type of drug stored in the memory of the RF tag 16.

Information from the RF tag 16 attached to the drug cartridge 10 is read by the RF-ID reader 277 by, for example, the following procedure. First, a signal for activation is generated from the transmission/reception circuit 279 of the RF-ID reader 277 to generate electric power to cause the RF tag 16 to transmit information to the RF tag 16, and electromagnetic waves are transmitted from the first portion 278A and/or the second portion 278B of the antenna 278. When the antenna 16A of the RF tag 16 receives electromagnetic waves, the antenna 16a receives the electromagnetic waves, thereby generating an electromotive force by resonance. The generated electromotive force activates the IC 16b, which reads the information stored in the storage section and converts it into a signal. The temperature detected by the first temperature sensor is converted into a signal. The generated signal is transmitted from the IC 16b to the antenna 16a, and electromagnetic waves are transmitted from the antenna 16a. The RF-ID reader 277 receives electromagnetic waves from the RF tag 16 by the first portion 278A or the second portion 278B of the antenna 278, and converts the electromagnetic waves into a signal by the transmission/reception circuit 279. Thus, drug information is obtained, including the first temperature information and information indicating the type of drug stored in the memory of the RF tag 16. These information are transmitted to the control device 280.

### Piston Drive Mechanism 220

Reference is made to FIG. 14 and FIG. 15. The piston 210 includes a distal end section 211 and a body 212 connected to the distal end section 211. The distal end section 211 is located on the first end 210a side. In the present embodiment, the distal end section 211 has an I-cut shape which is obtained by cutting a cylinder in two parallel planes along its axis. The distal end section 211 has a shape corresponding to the cap opening 130d provided in the cassette cap 130 of the cassette 100.

The piston 210 includes a drive protruding portion 213 located on the side surface of the body 212. The drive protruding portion 213 engages with a guide 231 provided in the piston drive mechanism 220 as will be described below, thereby regulating rotation based on the shape of the guide 231. In the present embodiment, the drive protruding portion 213 is a rib provided on the side surface of the piston 210, and the rib is a ridge-shaped protruding portion that extends parallel to the axis of the piston 210. In the present embodiment, the piston 210 has two drive protruding portions 213 arranged on the second end 210b side of the side surface of the body. The body 212 of the piston 210 has a female thread 214 located inside a hole 210h extending along the axis of the piston 210.

The piston drive mechanism 220 includes a motor 264, a gearbox 221, a drive gear 222, a drive rod 235 and a piston guide 230. The motor 264, the gearbox 221, the drive gear 222 and the piston guide 230 are supported by the internal housing 202.

The motor 264 rotates forward or rotates reverse based on the control by the control device 280. Herein, forward rotation refers to rotation in a direction that moves the piston 210 forward, and reverse rotation refers to rotation in a direction that moves the piston 210 backward.

The rotary encoder 265 is attached to the rotary shaft of the motor 264 as an amount of rotation detector, and the rotary encoder 265 detects the amount of rotation (number of revolutions) of the motor 264. The rotary encoder 265 includes an encoder plate 266 and a pulse encoder 267 that includes a light-emitting element and a light-receiving element. The rotary encoder 265 will be described in detail below.

The gearbox 221 includes at least one gear attached to the rotary shaft of the motor 264. The gearbox 221 may include two or more gears to reduce the rotational speed of the motor 264.

The drive gear 222 meshes with the gear of the gearbox 221 and is rotatably supported by the bearing 223 in the internal housing 202. A hole is provided in the shaft of the drive gear 222, into which one end of the drive rod 235 is inserted and fitted.

The drive rod 235 has a rod shape and has a male thread 236 on the side surface. The male thread 236 is configured in terms of thread height, shape, thread pitch, etc., to engage with the female thread 214 provided on the piston 210.

The piston guide 230 has a hole 230h into which the piston 210 is inserted. A guide 231 is provided on the inner side surface of the hole 230h. The guide 231 engages with the drive protruding portion 213 of the piston 210 and guides the piston 210 so that the piston 210 moves forward or backward without rotating around the axis.

In the present embodiment, the guide 231 is a straight groove extending parallel to the axis of the hole 230h, into which a rib, being the drive protruding portion 213, provided on the side surface of the piston 210 is inserted. The piston guide 230 includes two guides 231 corresponding to two drive protruding portions 213 of the piston 210.

When the motor 264 rotates forward by an instruction of the control device 280, the drive gear 222 rotates via the gearbox 221 and the drive rod 235 rotates. As the drive rod 235 rotates, the female thread 214 of the piston 210, which meshes with the male thread 236 of the drive rod 235, receives a rotational force around its axis. Since the drive protruding portion 213 of the piston 210 is inserted into the groove of the guide 231, the rotation of the piston 210 about its axis is restricted by the guide 231, and the piston 210 moves forward without rotation as the drive rod 235 rotates.

When motor 264 rotates reverse by an instruction of the control device 280, the drive gear 222 rotates via the gearbox 221, causing the drive rod 235 to rotate in the reverse direction. The male thread 236 of the drive rod 235 meshes with the female thread 214 of the piston 210, regulating rotation around the axis, thereby causing the piston 210 to move backward without rotating around its axis.

### Control of Piston Drive Mechanism 220

As described above, the viscosity of the liquid drug 14 varies depending on the temperature. Generally, the higher the temperature of the drug, the smaller the viscosity. Also, the viscosity varies depending on the type of drug. Moreover, even if the same drug is housed in a drug cartridge, the load required to move the gasket 13 may vary with different diameters of the cylinder 11 due to the variation of the capacity of the cylinder 11. With conventional drug injection devices, in order to ensure smooth injection operation regardless of the type of drug and the capacity of the drug cartridge 10, a sufficiently high drive voltage is supplied to the motor that can accommodate the most viscous drug among all compatible drugs, and the drug cartridge with the largest load among all compatible drug cartridges. However, this requires the use of a secondary battery with a large maximum output. This also increases the volume of the secondary battery, making the drug injection device larger and heavier. Since the motor is always driven by more electric power than necessary, the power consumption increases.

Conventional drug injection devices addressed the problem that injecting cold drug may cause pain by leaving the drug cartridge in the room for a certain period of time before the operator performs injection. Such a time management as to leave the drug cartridge for a certain period of time is troublesome for the operator, and since the temperature of the room at the time of injection can vary depending on the season, location, etc., the drug cartridge may not necessarily reach the same temperature even if it is left for the same amount of time.

In the present embodiment, the RF-ID reader 277 receives the drug information including information indicating the type of drug in the drug cartridge transmitted from the RF tag 16 of the drug cartridge and the first temperature information detected by the first temperature sensor, and the drive power of the motor based on the first temperature information is determined, and the motor driver is controlled so that the determined drive power is output. If the temperature indicated by the first temperature information is in a certain temperature range, it is determined that the temperature is not suitable for injection, and the injection operation is not started by not driving the motor 264.

In order to perform such control, control information for controlling the motor 264 for each of the plurality of drugs is stored in the memory 254. The control information for each drug includes a set of control parameters for the motor for each of the multiple temperature ranges.

Table 1 shows an example of a set of control parameters for the motor stored in the memory 254. Drug A and Drug B are shown as examples of the plurality of drugs. The viscosity of Drug B is higher than that of Drug A. Also shown are a plurality of temperature ranges: less than 0° C., 0° C. or more and less than 10° C., 10° C. or more and less than 20° C., 20° C. or more and less than 38° C., and 38° C. or more. 0° C., 38° C. and 10° C. are examples of the first temperature, the second temperature and the third temperature, respectively. When the drug cartridge 10 is not inserted in the cassette 100, the RF-ID reader 277 is unable to obtain either the drug information or the first temperature information. The memory 254 may further include a set of control parameters for the motor in the absence of such a drug cartridge.

TABLE 1

| | | | | | |
|---|---|---|---|---|---|
| Type of drug cartridge | Motor control parameters | | | | |
| | less than 0° C. | 0° C. or more less than 10° C. | 10° C. or more less than 20° C. | 20° C. or more less than 38° C. | 38° C. or more |
| Drug A | Drive Infor mation C | Drive information A1 | Drive information A2 | Drive information A3 | Drive information C |
| Drug B | Drive Information C | Drive information B1 | Drive information B2 | Drive information B3 | Drive information C |
| No drug cartridge | Drive information C | | | | |

If the first temperature information is less than 0° C., the drug may possibly be frozen. If the first temperature information is 38° C. or more, the drug may possibly be altered by being stored at a high temperature. If the first temperature information is in these two temperature ranges, it is not desirable to perform injection. Therefore, drive information C is assigned regardless of whether the drug type of the drug information is A or B. Drive information C may be, for example, information that the motor 264 is not to be driven. If the drug cartridge is not inserted into the cassette 100, injection cannot be performed with any temperature range, so drive information C is assigned with all temperature ranges. In the temperature ranges of 0° C. or more and less than 10° C., 10° C. or more and less than 20° C., and 20° C. or more and less than 38° C., sets of control parameters of drive information A1, A2, A3 and drive information B1, B2, B3 are assigned for drug A and drug B, respectively.

Table 2 shows a specific example of sets of control parameters of drive information A1, A2, A3 and drive information B1, B2, B3. The motor drive power may be adjusted, for example, by adjusting the voltage to be applied or by other means. In the present embodiment, the drive power is adjusted by PWM (pulse width modulation) to control the motor. FIG. 17 shows an example of a drive signal by PWM and the rotation speed of the motor that rotates by the drive signal. Drive information that defines the drive signal by PWM includes, for example, the duty ratio initial value, the duty ratio increment, the increase period, the increase maximum wait time and the target-achieving duty ratio.

For all of the temperature ranges, the viscosity of drug B is higher than that of drug A. Therefore, comparison between the temperature ranges shows that the initial value of the duty ratio initial value is greater for drug B than for drug A. This is because more electric power is needed to drive a drug with higher viscosity. In the present embodiment, the increase period and the increase maximum wait time are shorter for drug B than for drug A, and the target duty ratio is greater for drug B than for drug A for the temperature ranges.

For each drug, the values of the parameters are smaller for higher temperature ranges. That is, the higher the lower limit value of each temperature range, the smaller the duty ratio initial value, the duty ratio increment, the increase period, the increase maximum wait time and the target-achieving duty ratio become. This is because the higher the temperature of the drug, the smaller the viscosity and the lower the electric power required to drive the drug.

TABLE 2

| Type of drug | Drug A | | | Drug B | | |
|---|---|---|---|---|---|---|
| Drive information | A1 | A2 | A3 | B1 | B2 | B3 |
| (1) Duty ratio initial value (%) | 70 | 65 | 60 | 80 | 75 | 70 |
| (2) Duty ratio increment (%) | 5 | 4 | 3 | 4 | 3 | 2 |
| (3) Increase period (Encoder pulse count) | 5 | 4 | 3 | 4 | 3 | 2 |
| (4) Increase maximum wait time (ms) | 30 | 25 | 20 | 25 | 20 | 15 |
| (5) Target duty ratio (%) | 90 | 85 | 80 | 100 | 95 | 90 |

For example, if the drug information is drug A and the first temperature information is 15° C., the control device 280 reads out drive information A2 from the memory 254 and drives the motor driver 263 using the parameters included in drive information A2.

Regardless of which drive information is selected from among A1 to A3 and B1 to B3 as shown in Table 2 and FIG. 17, the control device 280 causes the motor driver to generate drive signals so that the current flowing to the motor increases in steps as the duty ratio increases in steps. When the duty ratio reaches the target duty ratio, the drive then continues at the target duty ratio. Thus, when starting the motor, the peak value (current overshoot) of the starting (start-up) current can be lowered by performing PWM control with an initial value of duty ratio in accordance with the viscosity of the drug and so that the duty ratio increases in steps.

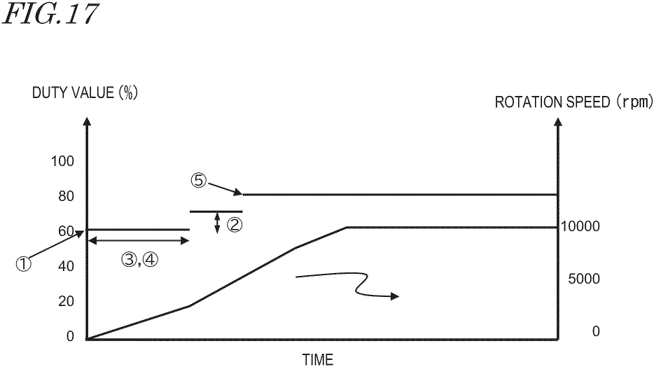

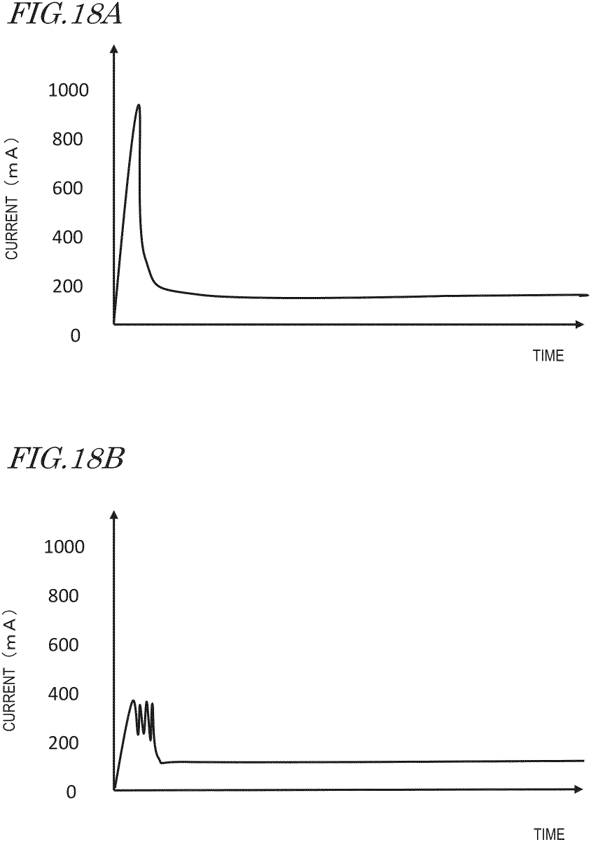

FIG. 18A shows the starting current that occurs when PWM control is not performed, and FIG. 18B shows an example of the starting current with PWM control in the present embodiment. As shown in FIG. 18A, when PWM control is not used, a peak current of about 800 mA at maximum flows. In contrast, according to the present embodiment, the peak current can be suppressed to about 400 mA.

Using an initial value of duty ratio according to the viscosity of the drug contributes particularly to reducing the amount of time until the motor 264 reaches a steady state. Even if the initial value of duty ratio is not changed according to the viscosity of the drug, the overshoot of the current at startup can be suppressed by driving the motor 264 using a pulse signal such that the duty ratio increases in steps. However, if an appropriate initial value of duty ratio according to the viscosity is not used, the amount of time required for the gasket of the drug cartridge 10 to start and move at a constant speed may become very long. This may increase the amount of time required for injection, making it impractical or placing a greater burden on the operator.

In the present embodiment, by setting the parameters of the overall PWM control, such as the duty ratio initial value, etc., taking into account the difference in viscosity due to the type of drug and the difference in viscosity due to temperature, the starting current can be reduced while preventing the amount of time until the motor 264 reaches a steady state from becoming long. This allows the maximum discharge current to be reduced, making it possible to reduce the capacity of the secondary battery. Thus, it is possible to realize a drug injection device that can perform injection with an appropriate drive force even with a smaller-capacity secondary battery. With the drug injection device of the present embodiment, the battery capacity can be reduced by about 20% to 30%, for example, as compared to the case where the drive power is not adjusted. Therefore, it is possible to realize a smaller and lighter drug injection device, which is easier to handle and provides excellent operability.

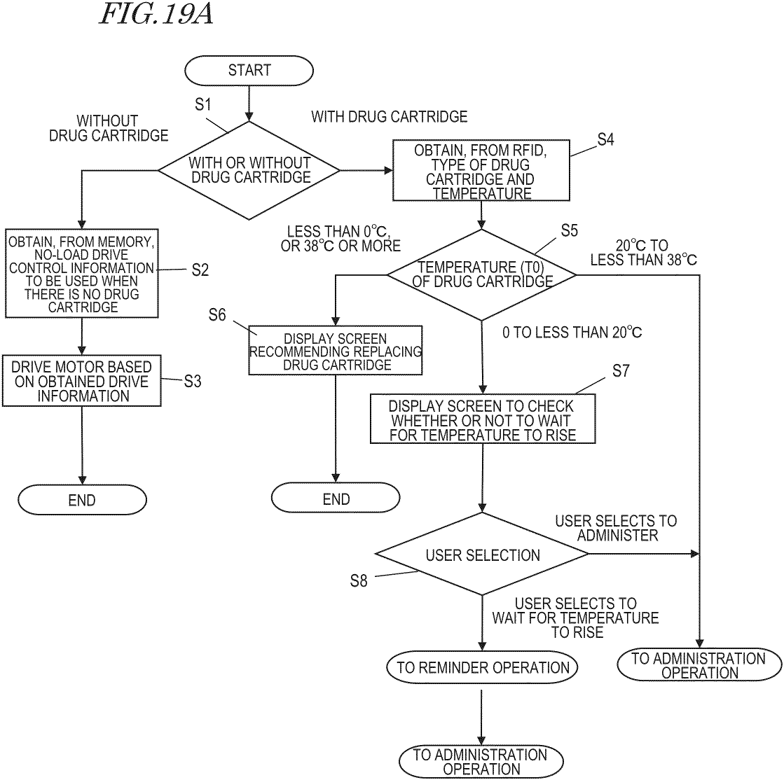

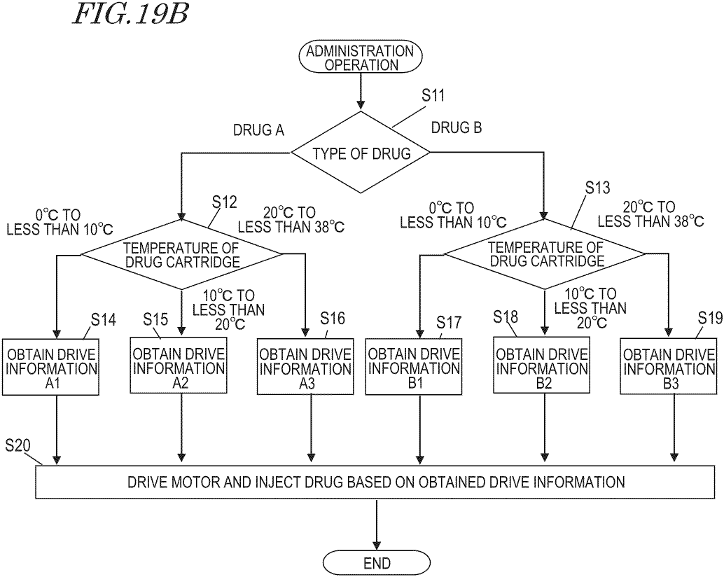

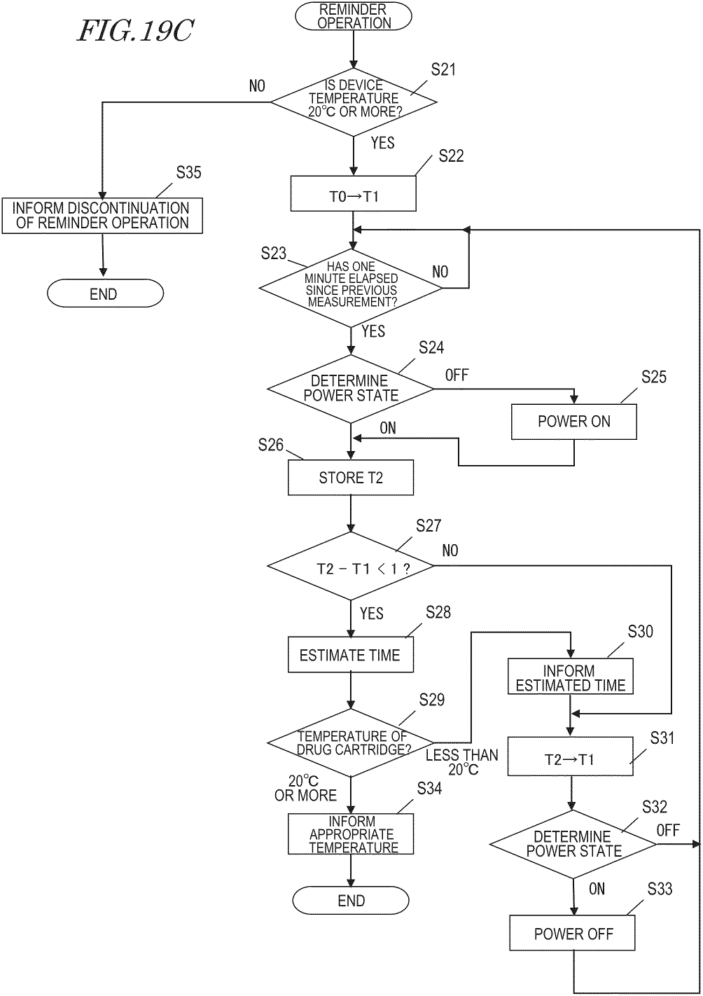

As described above, the first temperature information transmitted from the RF tag 16 of the drug cartridge 10 can be used also to determine whether the temperature is suitable for injection. Referring now to FIG. 19A and FIG. 19B, an example of an injection operation in which the first temperature information is used also to determine whether the temperature is suitable for injection will be illustrated. FIG. 19A and FIG. 19B are flow charts illustrating the operation of the drug injection device 200 during an injection operation. Referring to FIG. 5 and these figures, the injection operation will be described.

When the operator loads the cassette 100 in the drug injection device 200, the control device 280 causes the transmission/reception circuit of the RF-ID reader 277 to transmit a signal to generate an electromotive force to cause the RF tag 16 to transmit information (S1). If the RF-ID reader 277 cannot receive the signal from the RF tag 16, it means that the drug cartridge 10 is not inserted in the cassette 100. In this case, the control device 280 reads drive information C from the memory 254 (S2), and drives the motor 264 with drive information C (S3). Drive information C is, for example, information that controls the motor driver 263 so as to maintain the state where the motor 264 is stopped.

When the RF-ID reader 277 receives a signal from the RF tag 16 (S4), the control device 280 stores the drug information transmitted from the RF tag 16 of the drug cartridge 10, including information indicating the type of drug in the drug cartridge 10, and the first temperature information detected by the first temperature sensor, as T0, in memory 254. T0 is used also as the initial temperature to estimate the amount of time until the drug reaches an appropriate temperature as will be described below.

The control device 280 compares the temperature indicated by the obtained first temperature information with the reference temperature (S5). The reference temperatures are 0° C. (first temperature), 20° C. (third temperature) and 38° C. (second temperature). If the temperature indicated by the first temperature information is less than 0° C. or if it is 38° C. or more (first temperature range), the drug in the drug cartridge 10 may be frozen or altered due to the high temperature. Therefore, for example, the control device 280 causes the display device 259 to display a screen prompting to replace the drug cartridge 10 (S6), and ends the drug injection operation.

If the temperature indicated by the first temperature information is 0° C. or more and less than 20° C. (second temperature range), the temperature of the drug is low and pain may possibly be felt during injection. Therefore, for example, the control device 280 causes the display device 259 to display a screen prompting to wait for the temperature of the drug to rise before injection (S7). In this case, the operator may decide that he/she wants to inject the drug as soon as possible even if he/she may feel some pain, and therefore an input on whether to select injection or to wait is accepted (S8). If the operator selects to wait, the process proceeds to the flow of a reminder operation. On the other hand, if the operator selects injection, the process proceeds to the flow of an administration operation.

If the temperature indicated by the first temperature information is 20° C. or more and less than 38° C. (third temperature range), injection can be performed. Therefore, the process proceeds to the flow of an administration operation.

As shown in FIG. 19B, in the flow of the administration operation, the control device 280 first determines whether the drug information is drug A or drug B (S11). If the drug information is drug A, the first temperature information is compared with the reference temperature (S12), and drive information according to the comparison result is read out from the memory 254. As shown in Tables 1 and 2, drive information A1 is read from the memory 254 if the temperature indicated by the first temperature information is 0° C. or more and less than 10° C., drive information A2 is read from the memory 254 if the temperature is 10° C. or more and less than 20° C., and drive information A3 is read from the memory 254 if the temperature is 20° C. or more and less than 38° C. (S14, S15, S16). Similarly, when the drug information is drug B, the first temperature information is compared with the reference temperature (S13), and drive information according to the comparison result is read out from the memory 254. Drive information B1 is read out from the memory 254 if the temperature indicated by the first temperature information is 0° C. or more and less than 10° C., drive information B2 is read out from the memory 254 if the temperature is 10° C. or more and less than 20° C., and drive information B3 is read out from the memory 254 if the temperature is 20° C. or more and less than 38° C. (S17, S18, S19).

The control device 280 controls the motor driver 263 using the parameters of the selected drive information to rotate the motor 264 by PWM control as described above.

Next, the reminder operation is described. If the temperature of the drug is low and the operator selects to wait for the temperature of the drug to reach the appropriate temperature before injection, the control device 280 of the drug injection device 200 performs the reminder operation. FIG. 19C is a flow chart showing an example of the reminder operation.

In the reminder operation, the control device 280 successively receives the first temperature information from the first temperature sensor 15 attached to the drug cartridge 10 and informs when the drug temperature reaches the appropriate temperature. The present embodiment further estimates the amount of time required to reach the appropriate temperature and informs of the estimated time. For this, the control device 280 receives the second temperature information from the second temperature sensor 273 and successively obtains the first temperature information at predetermined time intervals when the second temperature information is equal to or greater than a predetermined temperature. Based on the obtained first temperature information and second temperature information, the control device 280 successively calculates the estimated time until it is possible to perform injection, and controls the display device to display information indicating the calculated estimated time.

Specifically, first, the control device 280 receives the second temperature information from the second temperature sensor 273 and determines whether the second temperature information is 20° C. or more (S21). The second temperature information output by the second temperature sensor 273 directly indicates the temperature inside the device housing 201. However, if the second temperature sensor 273 is less susceptible to heat generated by components, etc., in the device housing 201, the second temperature information is substantially identical to the environmental temperature at which the drug injection device 200 is used. Since the temperature of the drug rises to room temperature by taking the drug cartridge 10 out of a refrigerator, or the like, and keeping it in a room, if the room temperature is less than 20° C., it is difficult for the temperature of the drug to reach 20° C., i.e., the temperature suitable for injection, even when the drug cartridge 10 is kept in the room. Therefore, if the second temperature information is less than 20° C., for example, the control device 280 causes the display device 259 to display a screen informing that the room temperature is too low for injection (S35), and ends the reminder operation.

If the second temperature information is 20° C. or more, the control device 280 stores, as T1, temperature T0 of the drug cartridge that is first obtained (S22). The control device 280 adjusts the timing for obtaining the first temperature information. If one minute has elapsed since the first temperature information is obtained in the previous iteration or for the first time, the process proceeds to the next step (S23).

The control device 280 determines the status of the power of the drug injection device 200 (S24). If the power is OFF, e.g., due to auto power-off, the power is turned ON (S25), and the process proceeds to the next step. If the power ON, the process proceeds directly to the next step.

The control device 280 obtains the first temperature information and stores it as the current measured value T2 (S26). Then, the difference between T2 and T1, which is the measured value of the first temperature information in the previous iteration (one time before) is determined (S27). If the difference between T2 and T1 is 1 or more, it means the temperature of the drug is increasing rapidly and it is difficult to appropriately estimate the amount of time required for the drug to reach the appropriate temperature. Therefore, the process waits until a certain amount of time elapses again. Specifically, first, the latest first temperature information T2 is stored as T1 (S31), and the power status is determined (S32). If in auto power mode, the process turns OFF the power (S33) and returns to the step of waiting for one minute (S23). If not in auto power mode, the process does not change the power status and returns to the step of waiting for one minute (S23) .

If the difference between T2 and T1 is less than 1 (S27), the control device 280 calculates how much time is required for the temperature of the drug cartridge 10, in other words the temperature of the drug, to reach the appropriate temperature.

According to in-depth study by the present inventors, when the drug cartridge 10 that has been stored at a low temperature is held at the environmental temperature at which injection is performed, such as being stored in the room, the rate of temperature rise immediately after can vary depending on the size of the drug cartridge 10 and the amount of drug being held. However, it has been found that once the rate of temperature rise is decreases, the rate of temperature rise thereafter stays approximately the same, regardless of the size of the drug cartridge 10 and the amount of drug. More specifically, it has been found that when the difference between T2 and T1 is less than 1° C., the rate of temperature rise thereafter stays substantially the same regardless of the size of the drug cartridge 10 and the amount of drug, and the temperature of the drug can be approximated by the time elapsed since the time when the initial temperature TO was obtained.

For example, if the difference between T2 and T1 is less than 1° C., the temperature f(x) of the drug estimated from the elapsed time, where x denotes the elapsed time, is indicated by the following function, depending on the temperature range of the second temperature information, which can be said to be the environmental temperature.
(1) When the second temperature information is 25° C. or more and less than 30° C.: [Expression 1]
$$f \left(x\right) = 0.00005 {x}^{3} 0.04 {x}^{2} + 1.3 x + 1.6 \qquad \text{Equation (1)}$$
(2) When the second temperature information is 20° C. or more and less than 25° C.: [Expression 2]
$$f \left(x\right) = 0.00002 {x}^{3} 0.025 {x}^{2} + 0.96 x + 1.6 \qquad \text{Equation (2)}$$

For example, if the second temperature information is 25° C., the temperature of the drug after the time when the difference between T2 and T1 is less than 1° C. using Equation (1) can be estimated using the elapsed time. When the difference between T2 and T1 is less than 1° C., the elapsed time from the time when the initial temperature TO is obtained is denoted as tₙ. Where tₙ₊₁, tₙ₊₂, tₙ₊₃, . . ., tₙ₊ᵢ denote every minute elapsed from tₙ, and T2ₙ denotes the first temperature information T2 at tₙ, the estimated drug temperature T(i) at i minutes after tₙ is given by the following equation. [Expression 3]

$$T \left(i\right) = T {2}_{n} + \left(f \left({t}_{n + 1}\right) - f \left({t}_{n}\right)\right) \qquad \text{Equation (3)}$$

If the temperature of the drug suitable for injection is, for example, 20° C. or more, the control device 280 uses the elapsed time tₙ when the difference between T2 and T1 is less than 1 and Equation (1) to determine f (tₙ) . Also, f (tₙ₊₁ is determined where i=1. From these two values and the first temperature information T2ₙ, T (1) is determined. If T (1) is less than 20° C., the control device 280 similarly performs the calculation while increasing i by 1 until T(i) is 20° C. or more. When T(i) is 20° C. or more, the control device 280 uses i at that time as the estimated time.

The control device 280 determines whether the first temperature information T2ₙ at this time (the present time) is less than 20° C. (S29), and if it is less than 20° C., the control device 280 causes the display device 259 to display a screen informing of the estimated time, i.e., that the drug will reach the appropriate temperature after i minutes (S30). How the estimated time is informed is not limited to the display device 259, but the control device 280 may transmit the estimated time to the operator’s portable device, such as a smartphone or a tablet device, using the communication section 262, and the portable device may perform the informing function such as by displaying.

Then, the latest first temperature information T2 is stored as T1 (S31), and the power status is determined (S32). If in auto power mode, the process turns OFF the power (S33) and returns to the step of waiting for one minute (S23). If not in auto power mode, the process does not change the power status and returns to the step of waiting for one minute (S23). Moreover, the process repeats steps S23 to S29 to estimate the amount of time required to reach the appropriate temperature. In order to estimate the amount of time required to reach the appropriate temperature for the second and subsequent times, the elapsed time tₙ since the time at which the initial temperature TO is obtained and the first temperature information T2ₙ. By successively using the newest measured value, i.e., the first temperature information T2ₙ, for time estimation, it is possible to make more accurate estimation.

For time estimation for the second and subsequent times, the control device 280 determines whether the first temperature information T2ₙ is less than 20° C. (S29), and if it is 20° C. or more, the control device 280 causes the display device 259 to display a screen indicating that the appropriate temperature has been reached (S34), and the reminder operation is ended. Then, the process proceeds to the administration operation, as shown in FIG. 19A.

Table 3 shows an example calculation. Assume that the difference between T2 and T1 becomes less than 1° C. after 5 minutes (n=5) from the time when the initial temperature TO is obtained, and the first temperature information T2ₛ at this time is 13.45° C.

TABLE 3

| t | i | T2₅ | f(t₅) | f(t₅₊ᵢ) | T(i) |
|---|---|---|---|---|---|
| 5 | 0 | 13.45 | 7.1625 | 7.1625 | 13.45 |
| 6 | 1 | 13.45 | 7.1625 | 8.068 | 14.3555 |
| ... | | ... | ... | ... | ... |
| 14 | 9 | 13.45 | 7.1625 | 13.332 | 19.6195 |
| 15 | 10 | 13.45 | 7.1625 | 13.7875 | 20.075 |

The estimated drug temperature at i=1, i.e., after elapse of one minute since this point in time (after six minutes since obtaining the initial temperature TO), is 14.3555° C. Since the estimated drug temperature is 20.075° C., which is 20° C. or more, at i=10, it is determined that the estimated time to reach the appropriate temperature is 10 minutes after. As described above, if the time estimation is calculated again after one minute, the measured value T2₆ is used.

Thus, according to the present embodiment, the operator can decide how to use the time until the drug reaches the appropriate temperature, since the operator is informed how many more minutes it will take for the drug to reach the appropriate temperature and be ready for injection.

Note that, in the present embodiment, the estimated time until the drug reaches the appropriate temperature is calculated in the reminder operation, but the estimated time may not need to be calculated and informed. That is, the first temperature information may be obtained at predetermined time intervals, and when the first temperature information becomes equal to or greater than the predetermined temperature, it may be informed that the drug has reached the appropriate temperature and is ready for injection.

### Touch Sensor 275

As shown in FIG. 1 and FIG. 13, the touch sensor 275 is arranged on the skin-contact surface 201e of the protruding portion 201t of the drug injection device 200. Thus, it is possible to detect the state where the skin-contact surface 201e is reliably in contact with the skin, and to detect an inappropriate holding state such as wobbling of the drug injection device 200 before the needle 22 comes completely off the skin during injection. The touch sensor 275 will now be described in detail.

The touch sensor 275 is a touch sensor of a surface charge transfer type. The touch sensor 275 includes a sensor capacitor Cx arranged on the skin-contact surface 201e, a sampling capacitor Cs and a control circuit built in the control device 280. The control circuit of the touch sensor 275 first resets the touch sensor 275 by discharging the sensor capacitor Cx and the sampling capacitor Cs. Then, the control circuit of the touch sensor 275 charges the sensor capacitor Cx and transfers the charge charged in the sensor capacitor Cx to the sampling capacitor Cs. This charging and transferring operation is repeated to successively accumulate charge in the sampling capacitor Cs. The control circuit monitors the voltage of the sampling capacitor Cs, and when the voltage exceeds a predetermined value, the control circuit outputs the transfer count N representing the number of repetitions up to that point. The transfer count N is inversely proportional to the capacitance of the sensor capacitor Cx, and as the capacitance of the sensor capacitor Cx increases, the transfer count N decreases. When the skin-contacting surface 201e of the protruding portion 201t of the drug injection device 200 is in contact with the skin of the operator, etc., the capacitance of the sensor capacitor Cx increases, so the charge accumulated in the sensor capacitor Cx also increases and the amount of charge transferred to the sampling capacitor Cs in a single transfer operation also increases. Therefore, the transfer count N until the voltage of the sampling capacitor Cs exceeds a predetermined value decreases.

FIG. 20 shows an example of the results of checking the detection sensitivity of the touch sensor 275 using the drug injection device 200. In FIG. 20, the result of the value when N is about 900 times indicates the result when the skin-contact surface 201e is in contact with the skin, and the result of the value when N is about 1300 times indicates the result when the skin-contact surface 201e is not in contact with the skin. FIG. 20 shows that there is a large difference between the transfer count N when the skin-contact surface 201e is in contact with the skin and the transfer count N when it is not in contact with the skin, and that it is possible to clearly determine whether the skin is in contact by using the transfer count N. In the example described above, for example, if the transfer count is 1050 or less, it may be determined that it is in contact with the skin, and if the transfer count is 1100 or more, it may be determined that it is off the skin.

The time required for one transfer operation in a touch sensor of a surface charge transfer type is from 0.5 microsecond to 4 microseconds, for example, and the time required for the voltage of the sampling capacitor Cs to exceed a predetermined value is from about 2 ms to about 8 ms. The area of the skin-contact surface 201e is relatively small, and depending on the area where the injection is to be performed, it may be difficult to stabilize the attitude in which the drug injection device 200 is held during injection, and therefore when skin contact is detected five to eight times in a row, for example, it may be determined that the drug injection device 200 is correctly held and it is possible to perform injection. For example, if the touch sensor 275 detects a transfer count of 1050 or less for 5 consecutive times, it may be determined that the touch sensor 275 is detecting skin contact, and otherwise it may be determined that the touch sensor 275 detects separation from the skin.

A touch sensor of a surface charge transfer type is superior to conventional touch sensors of an oscillation type in that it is less susceptible to noise. Therefore, it is possible to detect that the skin is in contact with the skin-contact surface 201e more reliably than with conventional methods, and it may be possible to realize safer and more reliable handling of the drug injection device.

FIG. 21 is a flow chart illustrating the injection operation of the drug injection device 200 using the touch sensor 275. As shown in FIG. 21, after the start of the injection preparation operation, the control device 280 checks whether or not the touch sensor 275 is connected, etc., by the control circuit of the touch sensor 275 (S101), and if it is not connected, the control device 280 causes the display device 259 to display a fault or abnormality (S102).

If the touch sensor 275 is correctly connected, the display device 259 displays an image, text or other information that prompts the user to press the skin-contact surface 201e against the skin and hold the drug injection device 200 ready for injection (S103). The control device 280 successively receives detection signals from the control circuit of the touch sensor 275. For example, when a detection signal that indicates a transfer count of 1050 or less is received five consecutive times (hereinafter referred to as detecting skin contact, S104), the control device 280 causes the display device 259 to display information that prompts to press the inject button 258 (S105).

When the control device 280 detects a signal that the injection button 258 has been pressed (S106), the control device 280 controls the motor driver 263 so as to perform the injection operation described above, i.e., to inject the drug (S107). During the drug injection operation, the control device 280 receives successive detection signals from the control circuit of the touch sensor 275 in parallel. If skin contact cannot be detected, that is, if the touch sensor 275 detects separation from skin (S108), the control device 280 interrupts the injection operation and causes the display device 259 to display information indicating that the drug injection device 200 has come off the injection site and information that prompts to enter whether to discontinue or continue the injection (S110).

When the control device 280 detects a signal that the injection button 258 has been pressed (S111), the control device 280 confirms that the touch sensor 275 has detected skin contact (S112) and returns to injection operation (S113). If the operator makes an input to discontinue the injection (e.g., an input of the OK button 257), the control device 280 ends the injection operation.

The end of the injection operation is determined by the control device 280 based on the number of pulses or the number of revolutions detected by, for example, the rotary encoder 265 (S109), ending the injection operation.

### Acceleration Sensor 276

The drug injection device 200 of the present embodiment includes the acceleration sensor 276. Thus, it is possible to detect the attitude of the drug injection device 200 and to prompt the operator to change the attitude of the drug injection device 200.

As shown in FIG. 13, the acceleration sensor 276 can be arranged on the main board 290, for example. Preferably, the acceleration sensor 276 has first, second and third axes that are orthogonal to each other, and can detect acceleration along the axes or angular acceleration around the axes. It is also preferred that one of the first, second and third axes is arranged in the housing so that it coincides with the direction of movement of the piston. In the present embodiment, the acceleration sensor 276 detects acceleration along the first, second and third axes. For example, as shown in FIG. 22, in the drug injection device 200, the positive direction of the y axis coincides with the direction of movement of the piston 210 and the forward direction of the piston, and the direction of the normal vector of the plane on which the display device 259 is arranged is the z axis. Then, the x axis coincides with the direction of the normal vector of the right side surface of the drug injection device 200 when held in the upward orientation. The x axis, the y axis and the z axis are fixed to the drug injection device 200. Since the acceleration sensor 276 can detect gravitational acceleration along the x axis, the y axis and the z axis, it is possible to detect the attitude of the drug injection device 200, e.g., the tilt relative to the vertical direction of the y axis.

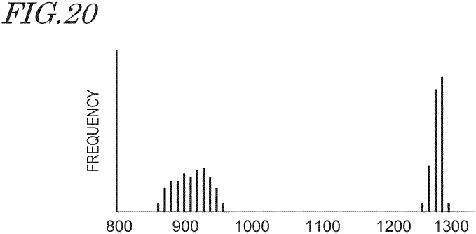

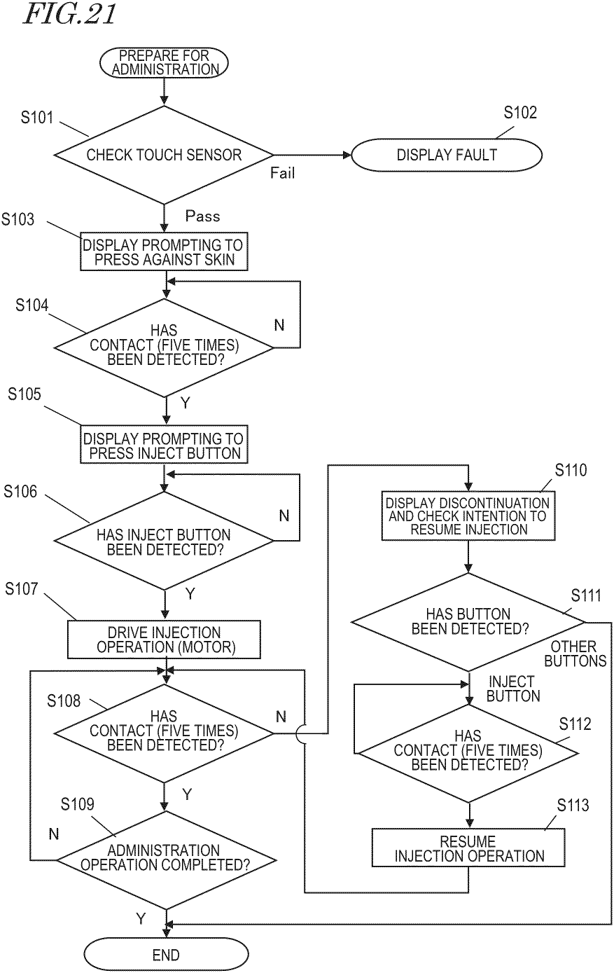

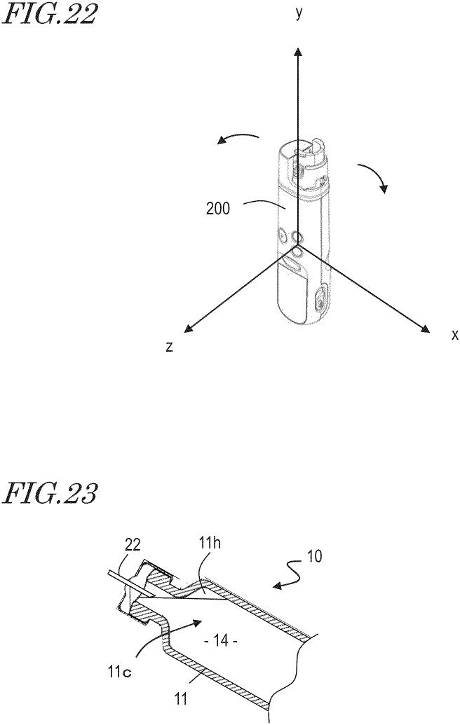

The result of such an attitude detection can be used for various operations and movements of the drug injection device 200. For example, when performing injection, it is preferred to perform air removal (also known as emptying) to remove air from inside the drug cartridge 10 in advance. This operation is performed by moving the piston 210 with the needle 22 pointing upward to expel air staying in an upper portion the drug cartridge 10 through the needle 22. However, if the distal end of the drug injection device 200 is tilted instead of pointing straight up, as shown in FIG. 23, air may stay in a corner 11h of the cylinder columnar space 11c, where air is difficult to escape, depending on the shape of the cylinder columnar space 11c of the drug cartridge 10. In such a case, the acceleration sensor 276 can be used to guide the operator to hold the drug injection device 200 in such an attitude that is suitable for air removal.

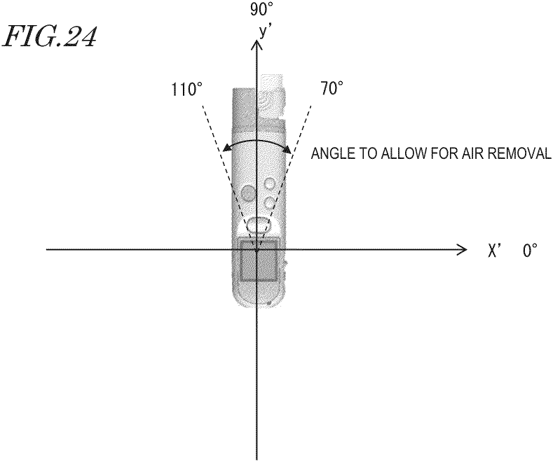

How much the drug injection device 200 should be held in an upright position without tilting when performing air removal depends on the cylinder columnar space 11c of the drug cartridge 10. As generally shown in FIG. 24, in coordinates where the vertical direction is fixed as the y′ axis, if the y axis of the drug injection device 200 is within ±70° from the vertical direction, i.e., in the range of 70° or more and 110° or less relative to the x′ axis, it is considered that the air can substantially be correctly expelled without the operator feeling difficulty in adjusting the attitude (adjusting the direction).

FIG. 25 shows a flow chart of an air-removing operation using the acceleration sensor 276. When the operator selects the air-removing operation, or when the control device 280 performs the air-removing operation after the operator inputs an instruction to perform injection, the control device 280 causes the display device 259 to display information that prompts the operator to hold the drug injection device 200 upward (information about the attitude of the drug injection device) (S201). The control device 280 receives the detection signal from the acceleration sensor 276, specifically, the acceleration information in the x-, y- and z-axis directions, calculates the angle of tilt of the y axis from the horizontal direction, and makes a determination regarding the tilt (S202). If the angle of tilt is an angle other than 70° to 110°, the drug injection device 200 is tilted, and the control device 280 causes the display device 259 to display information that indicates that the drug injection device 200 is tilted and prompts the operator to correctly hold the drug injection device 200 upward (S203).

If the calculated angle is 70° to 110°, the control device 280 controls the motor driver 263 to drive the motor 264 to move the piston 210 forward to start the air-removing operation (S204).

While the piston is moving, the control device 280 successively receives the detection signal from the acceleration sensor 276 and makes a determination regarding the tilt (S205). If the angle of tilt becomes an angle other than 70° to 110°, the control device 280 stops driving the motor 264 and causes the display device 259 to display information that indicates that the drug injection device 200 is tilted and prompts the operator to correctly hold the drug injection device 200 upward (S207). Moreover, when the angle of tilt becomes 70° to 110° (S208), the drive of the motor 264 is restarted (S208).

The end of the air-removing operation is determined by the control device 280, for example, based on the number of pulses or the number of revolutions detected by the rotary encoder 265 (S206), ending the air-removing operation.

Thus, by using the acceleration sensor 276 to detect the attitude of the drug injection device 200, information regarding the more appropriate handling of the drug injection device 200 can be presented to the operator.

As described above, the drug injection device 200 of the present embodiment is also compatible with the drug cartridges 10′ containing drug in which the liquid component and the solid component are held separately in an unused state. When using the drug cartridge 10′, the liquid component 14A and the solid component 14B are brought into contact with each other before use, as described above with reference to FIG. 9A, FIG. 9B, FIG. 10(a) and FIG. 10(b). However, simply bringing the liquid component 14A and the solid component 14B into contact may not cause the solid component 14B to dissolve immediately into the liquid component 14A. Therefore, it is preferred for the operator to move the drug injection device 200 to mix (stir) the liquid component 14A and the solid component 14B. The detection of the attitude of the drug injection device 200 by the acceleration sensor 276 is also suitable for performing such a mixing operation. The drug mixing operation using the acceleration sensor 276 will now be described.

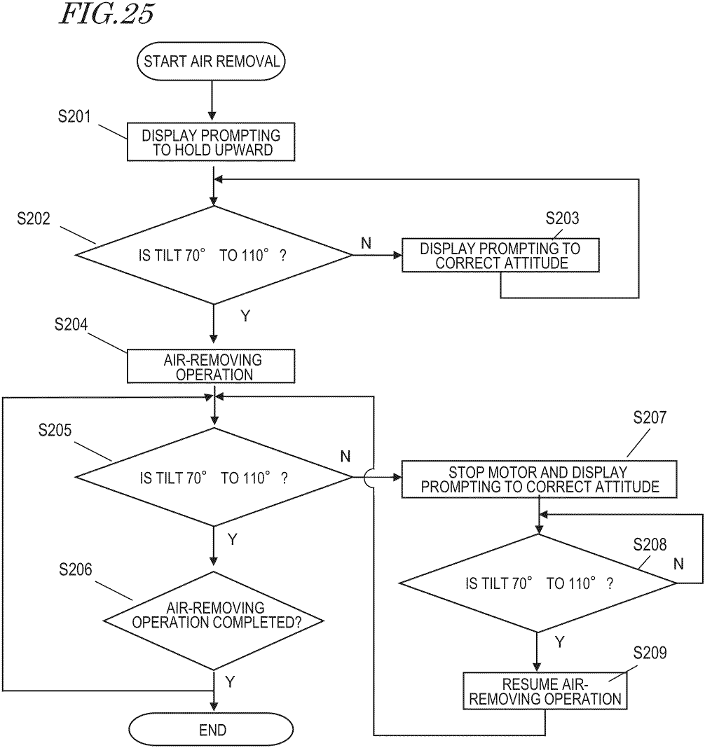

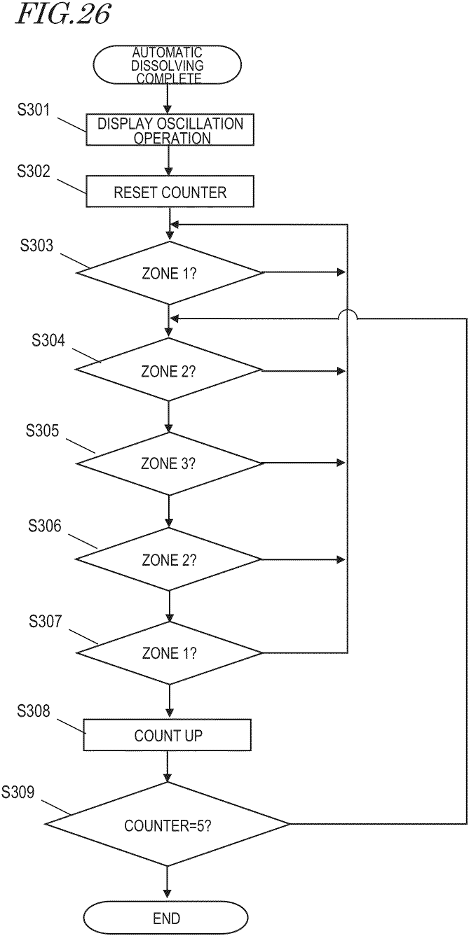

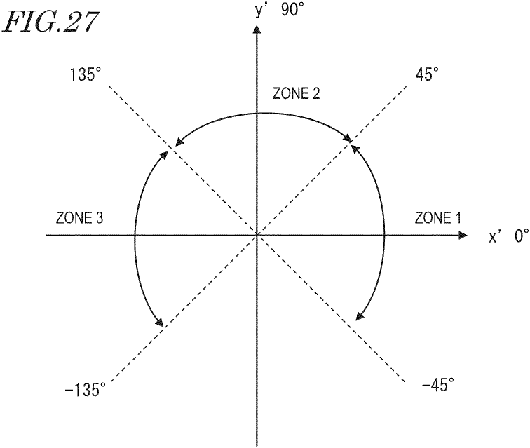

FIG. 26 is a flow chart of the mixing operation. To move the drug, one may consider oscillating the drug injection device 200 up and down, for example. However, such an operation may cause the liquid component 14A to foam easily, making it impossible to perform injection immediately after mixing. In addition, moving the drug injection device 200 up and down may possibly cause the drug injection device 200 to hit furniture such as a desk, which is undesirable. In the present embodiment, in order to slowly mix the drug without foaming, the drug injection device 200 is tilted left and right as indicated by arrows in FIG. 22. The attitude of the drug injection device 200 is indicated by the angle of the y axis of the drug injection device 200 shown in FIG. 22 on the x′y′ coordinates shown in FIG. 27. The x′y′ coordinates are fixed regardless of the attitude of the drug injection device 200. As shown in FIG. 27, it is represented by ±180° with 0° being the positive direction of the x′ axis. As shown in FIG. 27, the range from -45° to 45° is defined as Zone 1, the range from 45° to 135° as Zone 2, and the range from -180° to -135° or from 135° to 180° as Zone 3.

The control device 280 determine that the operator is correctly oscillating the drug injection device 200 and mixing the drug by determining that the angle of the y axis of the drug injection device 200, as calculated using the acceleration sensor 276, falls within the range of Zone 1, the range of Zone 2 and the range of Zone 3 in a predetermined order.

As shown in FIG. 26, first, after bringing the liquid component 14A and the solid component 14B into contact before use (automatic dissolving operation), the control device 280 causes the display device 259 to display information that prompts the operator to perform the oscillating operation described above (S301). Then, the counter is reset to count the number of oscillations (S302). The operator tilts the drug injection device 200 left and right in the order of P1, P2, P3, P4 and P1, as shown in FIG. 28. In this operation, the control device 280 receives a detection signal from the acceleration sensor 276 and successively determines that the y-axis tilt is in the following ranges: Zone 1 (-45° to 45°) (S303), Zone 2 (45° to 135°) (S304), Zone 3 (-180° to -135° or 135° to 180°) (S305), Zone 2 and Zone 1 in this order.

Then, the control device 280 increments the count by one and determines whether the number has reached a specified value (S309). For example, the specified value is five, and if the count is less than or equal to the specified value, the detection operation described above is repeated (S303 to S308) .

If it does not fall within the zones, it is determined that correct oscillation is not being performed, and the detection of the attitude is redone from the state where the attitude has returned to Zone 1. When the count reaches the specified value (S309), the mixing operation is ended.

By performing such an operation, the control device 280 can detect that the operator has performed the mixing operation correctly.

Note that according to the control of the mixing operation described above, one reciprocal oscillation is not counted unless the attitude of the drug injection device 200 is not detected to enter Zone 1 and Zone 3, particularly. Therefore, if the operator repeats improper oscillation operation, it may possibly become difficult to complete the oscillation operation. In such a case, images corresponding to the operations denoted as P1 to P4 in FIG. 28 may be sequentially displayed on the display device 259. For example, images showing the operations from P1 to P4 may be displayed before determination in S303, S304, S305, S306 and S307 in the flow chart. Such a display makes it easier for the operator to move the drug injection device 200 in accordance with the display, thereby preventing improper operations such as small tilt angles. By receiving specific operation instructions, the operator can confirm that the operation is not incorrect, that the movement is appropriate, etc., and can use the drug injection device 200 with less worry.

### Rotary Encoder 265

The rotary encoder 265 detects the number of revolutions (the amount of driving) of the motor 264. Since the amount of drug to be injected is set by the number of revolutions of the motor 264, it is important that the number of revolutions of the motor 264 be accurately measured, and it is therefore important that the rotary encoder 265 be capable of accurate measurement. The rotary encoder 265 of the present embodiment has a structure with which it is possible to correctly detect a fault when the rotary encoder 265 itself fails.

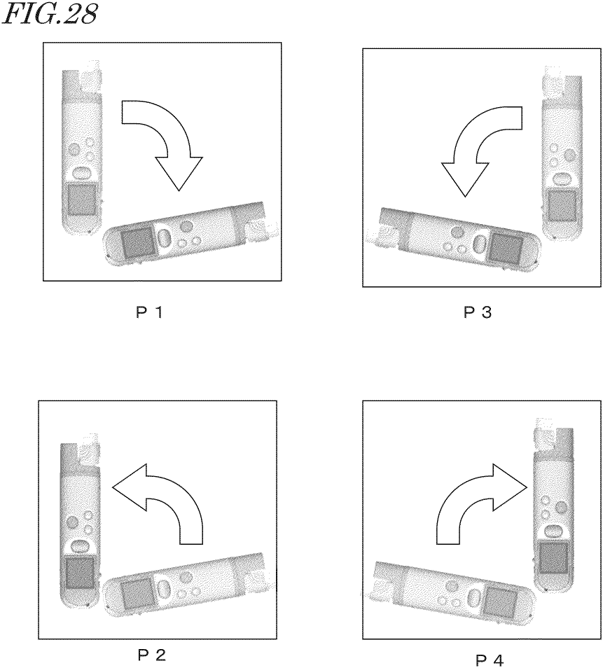

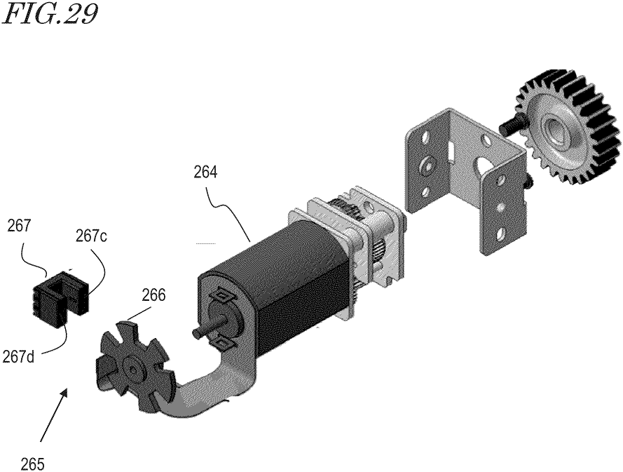

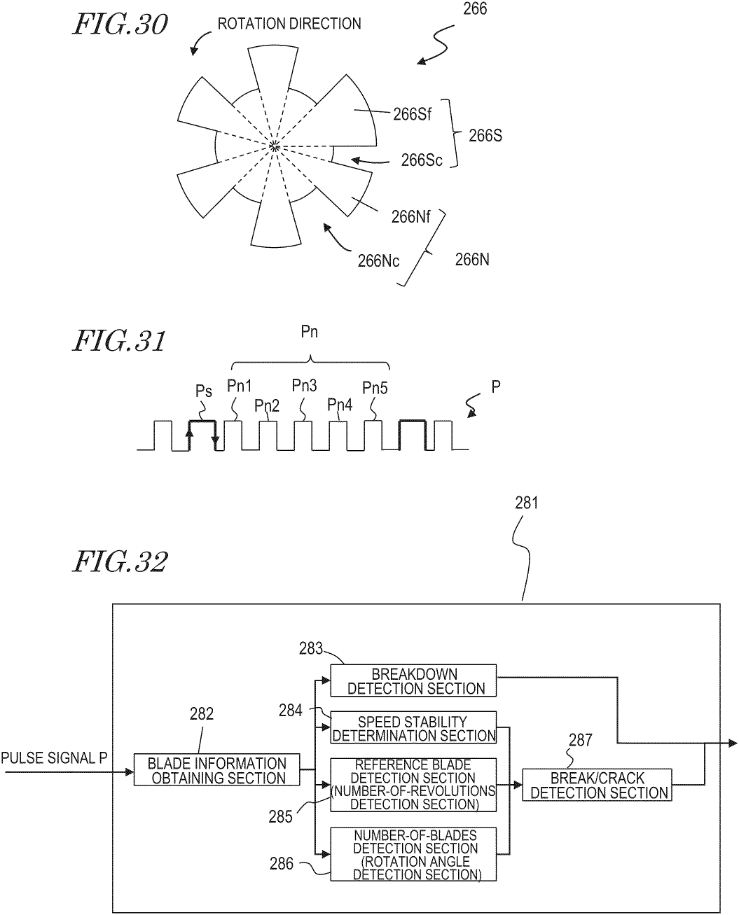

FIG. 29 is an exploded perspective view of a portion of the piston drive mechanism 220 including the rotary encoder 265. As described above, the rotary encoder 265 includes the encoder plate 266 attached to the rotary shaft of the motor and the pulse encoder 267 including a light-emitting element 267c and a light-receiving element 267d. FIG. 30 is a plan view of the encoder plate 266. The encoder plate 266 includes one reference blade section 266S and a plurality of normal blade sections 266N arranged along the circumference. In the present embodiment, the encoder plate 266 includes five normal blade sections 266N. The reference blade section 266S includes a blade 266Sf and a notch 266Sc. Each normal blade section 266N includes a blade 266Nf and a notch 266Nc.

The lengths of the blades 266Nf of the normal blade sections 266N in the circumferential direction are equal to each other. The lengths of the notches 266Nc of the normal blade sections 266N in the circumferential direction are equal to each other. In contrast, the length of the blade 266Sf of the reference blade section 266S in the circumferential direction and the length of the notch 266Sc in the circumferential direction are different from the length of the blade 266Nf of the normal blade section 266N in the circumferential direction and the length of the notch 266Nc of the normal blade section 266N in the circumferential direction, respectively.

In the present embodiment, the center angles of the blade 266Nf and the notch 266Nc of the normal blade section 266N are 30°. On the other hand, the center angle of the blade 266Sf of the reference blade section 266S is 45°, and the center angle of the notch 266Sc is 15°. By making the center angle of the blade 266Sf of the reference blade section 266S about 1.5 times the center angle of the blade 266Nf of the normal blade section 266N, it is easier to detect the difference between the reference blade section 266S and the normal blade section 266N. The larger the blade 266Sf is, the smaller the notch 266Sc becomes, but the notch 266Sc can be detected with sufficient accuracy as long as the difference is about 1.5 times.

The encoder plate 266 is attached to the rotary shaft of the motor 264, and when the motor makes one revolution, the encoder plate 266 also makes one revolution.

The light-receiving element 267d of the pulse encoder 267 is arranged in a position at which light emitted from the light-emitting element 267c enters. The pulse encoder 267 generates a pulse signal by detecting changes in the amount of light as the reference blade section 266S and the normal blade sections 266N of the encoder plate 266, which rotates in sync with the rotation of the motor 264, cross the optical path between the light-emitting element 267c and the light-receiving element 267d. FIG. 31 shows an example of a pulse signal. The example shown in FIG. 31 shows a signal obtained when the encoder plate 266 shown in FIG. 30 rotates counterclockwise. The pulse signal P includes a pulse Ps corresponding to the blade 266Sf of the reference blade section 266S and pulses Pn corresponding to the blades 266Nf of multiple normal blade sections 266N. The rising edge of each pulse is called the control edge and the falling edge is called the check edge. Control edges and check edges can be distinguished based on whether the light intensity detected by the photodetector increases or decreases.

The control device 280 receives the pulse signal P from the rotary encoder 265 and controls the motor driver 263 based on the pulse signal P. Specifically, the control device 280 controls the number of revolutions of the motor 264 based on the pulse signal P. It also detects a fault of the rotary encoder 265 and stops the rotation of the motor 264 when a fault is detected. One of the main faults of the rotary encoder 265 that should be detected is breaking or cracking of a blade of the encoder plate 266. A broken blade means that the blade breaks near the base and drops off. When a blade breaks, the number of pulses generated per revolution of the encoder plate 266 decreases. Conversely, when a blade is cracked, the blade generates two or more pulses, so the number of pulses generated per revolution of the encoder plate 266 increases. In either case, these faults should be detected early because they cause errors in the measurement of the number of revolutions and the angle of rotation of the motor 264 and affect the amount of drug administered.

For this, the control device 280 includes a fault detector 281, as shown in FIG. 32. The fault detector 281 includes a blade information obtaining section 282, a breakdown detection section 283, a speed stability determination section 284, a reference blade detection section 285, a number-of-blades detection section 286, and a break/crack detection section 287.

The blade information obtaining section 282 receives the pulse signal from the pulse encoder 267 to measure the times of the control edge and the check edge of the pulses Ps and the pulses Pn in the pulse signal P based on the reference clock of the control device 280, for example.

The breakdown detection section 283 successively calculates the blade ratio and outputs a detection signal indicating an abnormality when the blade ratio becomes less than or equal to a reference value. Specifically, the breakdown detection section 283 detects an abnormality in which one or more blades 266Sf or 266Nf are broken down and missing the encoder plate 266. If one or more blades are completely missing, the number of pulses per revolution is more definitely reduced than if a part of a blade is broken and missing, and there will be a greater error in the amount of drug administered. The blade ratio is the ratio of the width of a pulse (Ps, Pn) in the pulse signal P with respect to the period of time from the control edge of the pulse to the control edge of the next pulse, i.e., the ratio of the time width from the control edge to the check edge of the pulse. As shown in Table 4, the blade ratio of the pulse Ps is 75% (75/100) and the blade ratio of the pulse Pn is 50% (50/100). If one blade adjacent to the normal blade section 266N (e.g., the blade 266Nf corresponding to the pulse Pn2 adjacent to the pulse Pn1) is missing, the blade ratio is 25% (50/200), and if one blade adjacent to the reference blade section 266S (the blade 266Nf corresponding to pulse Pn1) is missing, the blade ratio is 37.5% (75/200). If two blades adjacent to the normal blade section 266N are missing, the blade ratio is 16.7% (50/300), and if two blades adjacent to the reference blade section 266S are missing, the blade ratio is 25% (75/300). Thus, where 37.5% is used as the reference value and the blade ratio is less than or equal to 37.5%, for example, it may be determined that the blades of one or more blade portions are broken down.

The reference value used in the determination by the breakdown detection section 283 to may be another value. If the reference value is set to a value smaller than 25%, e.g., if the reference value of 24%, it may be determined that two or more blades are consecutively missing. If two or more blades are consecutively missing, the number of pulses per revolution of the encoder plate 266 will be even smaller, and the error in the amount of drug administered will be even larger. The reference value to be set is not limited to the values shown in Table 4, and the reference value may be determined by taking into consideration the margin due to variations in the rotational speed of the motor 264.

TABLE 4

| | No breakdown | Where adjacent blades are broken down | |
|---|---|---|---|
| | | Number of blades broken down | |
| | | 1 | 2 |
| Blade ratio of reference blade section | 75% | 37.5% | 25% |
| Blade ratio of normal blade section | 50% | 25% | 16%, |

Each time the control edge of a pulse Ps, Pn is detected, the breakdown detection section 283 calculates the blade ratio using the time of the control edge and the time of the check edge of the immediately preceding pulse, and if it is a value in the range described above, the breakdown detection section 283 outputs a signal indicating a fault. As described above, a breakdown of a blade is likely to leads to a decrease in the number of pulses and gives a large error in the measurement of the number of revolutions and the angle of rotation of the motor 264. Therefore, if the breakdown detection section 283 outputs a signal indicating an abnormality, the control device 280 directly controls the motor driver 263 to stop the motor 264 immediately, without using the determination result of the speed stability determination section 284 to be described below.

The speed stability determination section 284 determines whether the rotational speed of the encoder plate 266 is stable or not. This can be determined, for example, by measuring the length of the pulse interval (the time interval between the control edges of two adjacent pulses). However, if blades are broken or cracked, the pulse interval will vary. Specifically, if a part of a blade is missing, the pulse width may be shorter and the pulse interval may be longer. If a blade is broken, i.e., the blade is completely broken down and missing, the pulse interval will also be longer. If a blade is cracked, a single pulse turns into two pulses, thereby shortening the pulse interval in two places. That is, the pulse interval obtained in one revolution of the encoder plate 266 may possibly include those that have become longer or shorter due to chipping, breaking or cracking of a blade. Therefore, the speed stability determination section 284 excludes these and successively obtains and outputs the pulse interval average time.

If the number of blades of the encoder plate 266 is n and the blades are cracked, the number of pulses generated in one revolution is n+1 and the pulse interval between two pulses is short. When pulses are detected for r revolutions of the encoder plate 266, the number of short pulse intervals is (2/ (n+1) ) × (nr) . Similarly, when a blade is chipped, the number of long pulse intervals is (1 / (n-1) ) × (nr) .

Therefore, if the number of blades n is 6 and a pulse signal for 18 pulses (3 revolutions) is obtained, (2 / (6+1) ) × (18) = 5.14, which includes 5 to 6 short pulse intervals. Similarly, (1 / (6-1) ) × (18) = 3.6, which includes 3 to 4 long pulses.

Therefore, in order to obtain the correct rotation speed even when blades are cracked or broken, the pulse intervals for 18 pulses obtained are arranged in the order of length so as to exclude data for the four highest (long) pulse intervals and the six lowest (short) pulse intervals to determine the stability from the eight pulse intervals. For example, the standard deviation of the eight pulse intervals is determined, and if the standard deviation value is greater than a predetermined value, a signal indicating that the rotation speed is not stable is output. Alternatively, if the standard deviation value is smaller than a predetermined value, a signal indicating that the rotation speed is stable is output.

The reference blade detection section 285 detects a pulse Ps based on the reference blade section 266S in the pulse signal P to output a detection signal. The blade ratio described above can be used for the detection.

The number-of-blades detection section 286 detects pulses Ps and Pn in the pulse signal to output a detection signal.

The break/crack detection section 287 receives signals from the speed stability determination section 284, the reference blade detection section 285, and the number-of-blades detection section 286 to determine whether a blade is broken or cracked. For example, when a signal indicating that the rotation speed is stable is received from the speed stability determination section 284, the number of pulses per revolution is determined based on the detection signal output from the reference blade detection section 285 and the detection signal from the number-of-blades detection section 286. If the number of pulses is not six, it is determined that there is a blade that is broken or cracked (hereafter, a broken or cracked blade is referred to as a fault) to output a signal indicating a fault. If a signal indicating that the rotation speed is stable is not received from the speed stability determination section 284, or if a signal indicating that the rotation speed is not stable is received from the speed stability determination section 284, the detection signal from the number-of-blades detection section 286 may possibly be not accurate, and therefore the detection result is not output. Alternatively, the break/crack detection section 287 outputs a signal indicating that correct measurement is impossible. This can prevent false detection of a broken or cracked blade of the encoder plate 266 due to an unstable rotation speed of the motor 264. When the control device 280 receives a signal indicating a fault from the break/crack detection section 287, the control device 280 controls the motor driver 263 so as to stop the motor 264.

Note that the determination result of the speed stability determination section 284 is used for the determination of a fault in the number-of-blades detection section 286, but even if a signal indicating that the rotation speed is stable is output from the speed stability determination section 284, a fault may be erroneously detected due to the number of revolutions being stable. That is, there is some possibility of an erroneous fault detection in a single fault determination by the number-of-blades detection section 286. In such a case, the probability of erroneous fault detection can be made smaller by using the detection results of the break/crack detection section 287 a plurality of times. For example, one may consider setting the number of fault detections used in one determination to a plurality of times, and accumulating the number of times a fault is determined.

For example, N consecutive detection results may be used for one determination of the break/crack detection section 287, and the determination of the break/crack detection section 287 is accumulated M times.

Where it is demanded that the stability of the rotational speed of the motor 264 be high and the accuracy of the amount of drug administered be strict, the possibility of false detection of the break/crack detection section 287 is small to begin with. If there is a possibility that the amount of drug administered may not be accurate, it is preferable not to administer the drug. For this, it is preferred to determine a fault with N=1 and M=1. That is, if the break/crack detection section 287 detects a fault even once, it is preferred to stop the motor 264 or suspend the injection operation.

On the other hand, if there is a margin of accuracy in the amount of drug administered, and there is little possibility of adversely influencing the operator even if the amount administered is a little less or a little more, it is preferred to set N and M to values greater than 1 to further reduce the probability of false detection. For example, if N=2 and M=2, then a false detection is determined based on detection results of four iterations. Table 5 shows one such example. In Table 5, a fault (denoted as F in Table 5) is detected in the second and fourth iterations, but it is not determined as a fault (M is not counted) because a fault is not detected twice in a row (N=1). In the fifth and eighth iterations, a fault is detected twice consecutively, and it is determined that there have been two faults by accumulation in the eighth inspection. Therefore, in the eighth detection, a signal is output indicating that the break/crack detection section 287 has determined a fault.

TABLE 5

| Number of fault detections performed | 1 | 2 | 3 | 4 | 5 | 6 | 7 | 8 | 9 | 10 |
|---|---|---|---|---|---|---|---|---|---|---|
| Detection result | | F | | F | F | | F | F | F | F |
| N | 0 | 1 | 0 | 1 | 2 | 0 | 1 | 2 | 1 | 2 |
| M | 0 | 0 | 0 | 0 | 1 | 1 | 1 | 2 | 2 | 3 |

The fault detection of the rotary encoder 265 described above may be set to be performed whenever the drug injection device 200 is in operation and the motor 264 rotates. Alternatively, if it is undesirable for the injection to be interrupted or ended halfway through by detecting a fault and stopping the motor 264 while an injection is being performed, the fault detection may be not performed during the injection operation. Alternatively, the fault detection may be performed during the injection operation, but the motor 264 may be stopped after the injection is completed.

With either control, when the control device 280 detects a fault, the control device 280 causes the display device 259 to display information indicating the fault. This allows the operator to recognize the abnormality.

### User Interface

Information obtained from the various detection devices described above that are included in the drug injection device 200 can be displayed on the display device 259 or information for prompting the operator to operate the drug injection device 200 using the detection devices can be displayed on the display device 259. Example images that the control device 280 causes the display device 259 to display will now be described.

FIG. 33 shows an example of information of the RF tag 16 of the drug cartridge 10 to be displayed on the display device 259. As shown in FIG. 33, an image 310 includes a first region 310a, a second region 310b and a third region 310c. The first region 310a displays text information indicating the operation to be instructed to the operator or the status of the drug injection device 200. In the example shown in FIG. 33, the word “Information” is displayed because drug information stored in the RF tag 16 is being displayed. The second area 310b is colored with a theme color that corresponds to the type of the drug, for example. The third area 310c displays specific drug information.

In the present embodiment, for example, the drug is “XXXXX” and the second region 310b is colored in blue. The name of the drug, the dose per injection, and the amount of drug remaining (the number of times the drug can be injected) are displayed in the third area 310c, along with an image of the drug cartridge 10. The amount of drug remaining may be stored in the RF tag 16 or stored in the memory of the drug injection device 200. As the image of the drug cartridge 10 is displayed together, it is easier for the operator to recognize that the text information relates to the drug cartridge 10. The drug information shown in FIG. 33 is displayed, for example, after the cassette 100 is loaded and after performing the injection operation.

By using the format of the image 310 in other operations as well, it is easier for the operator to recognize what information is displayed where on the screen of the display device 259. In this case, the second area 310b should be colored in the theme color of the drug on the screen during any operation. Thus, the operator can recognize the type of drug being injected in any operation state.

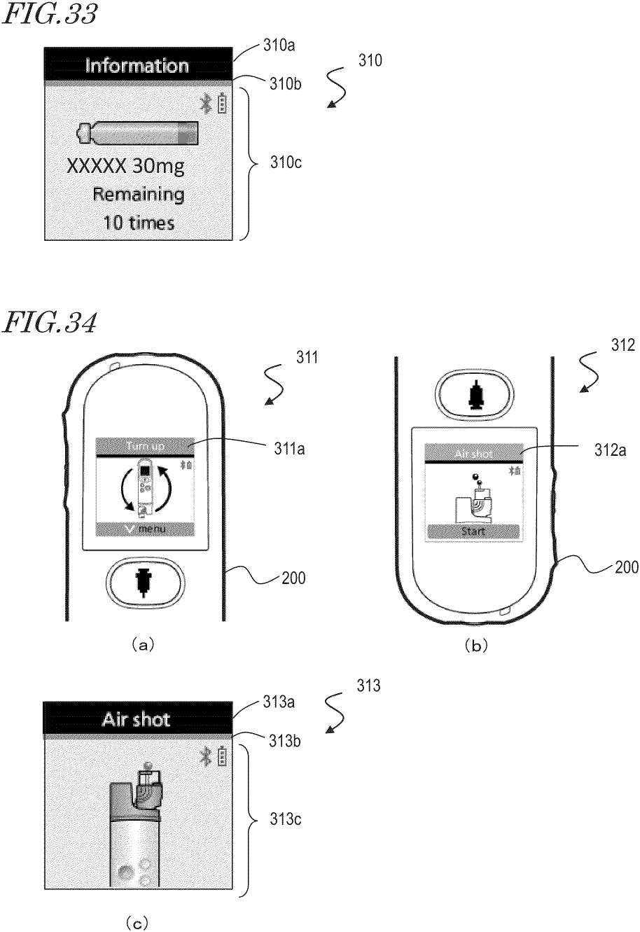

FIG. 34(a) to FIG. 34(c) show example images displayed in the air-removing operation using the acceleration sensor 276. FIG. 34(a) shows an image 311, which prompts the operator to invert and hold the drug injection device 200 before the air-removing operation. FIG. 34(b) shows an image 312 just before the start of the air-removing operation, with the operator holding the drug injection device 200 inverted and with the distal end facing up. In FIG. 34(a), the first area 311a is displayed on the rear end side of the drug injection device 200 on the display device 259, while in FIG. 34(b), the first area 312a is displayed on the distal end side of the drug injection device 200 on the display device 259. That is, when the drug injection device 200 is held inverted, the control device 280 reverses the orientation of the image 312 displayed on the display device 259 based on the detection signal from the acceleration sensor 276. By matching the orientation of the image 312 displayed on the display device 259 to the attitude of the drug injection device 200, it is possible to present information that is easily recognized by the operator.

Then, while the control device 280 is performing the air-removing operation, the control device 280 may display an image 313 with an animation showing the air removal being performed in the third area 313c as shown in FIG. 34(c).

FIG. 35(a) and FIG. 35(b) are example images 314 and 315 that prompt the operator to remain in the same state until the operation of the drug injection device 200 is complete. Since the length of time to wait is not constant, the waiting time is not displayed, but an animation is shown in which designs arranged in a circular arrangement appear to be moving by blinking the designs. By thus displaying the animation, the operator can recognize that the operation of the drug injection device 200 is normal.

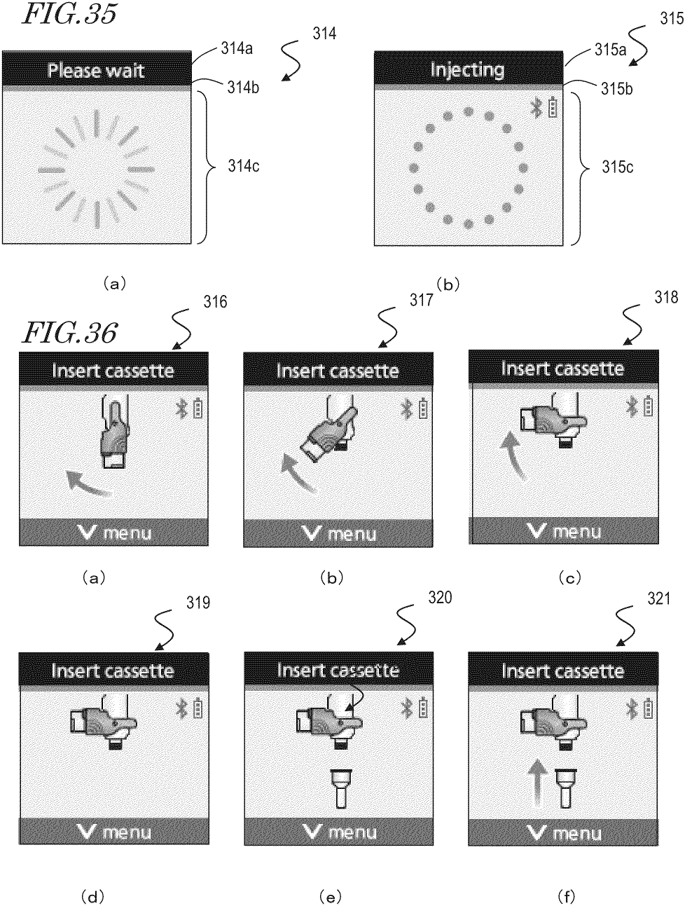

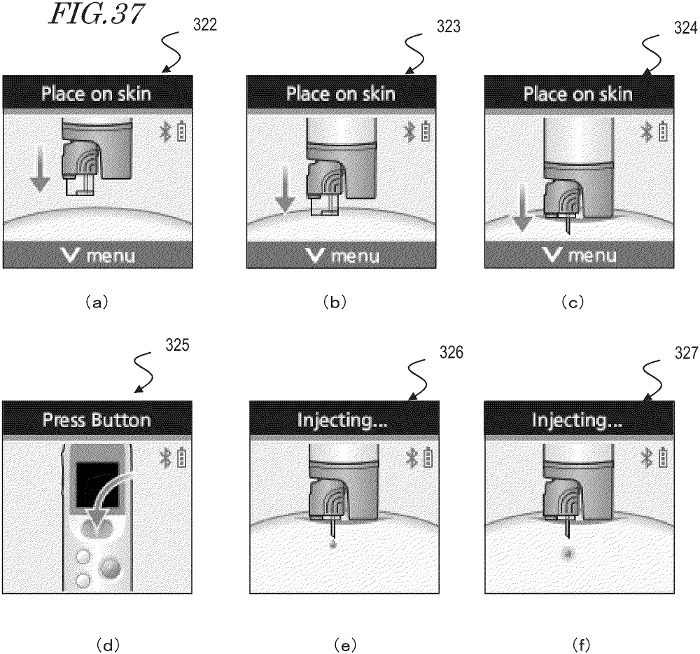

FIG. 36(a) to FIG. 36(f) show example images 316 to 321 that prompt the operator to attach the needle unit 20 and remove the needle case 25 after attachment. By sequentially repeatedly displaying the images shown in FIG. 36(a) to FIG. 36(f) until the operator removes the needle case 25, the operator can easily understand how to operate and how far the operator should proceed in the operation.

FIG. 37(a) to FIG. 37(f) show examples images 322 to 327 that prompt the operator to press the drug injection device 200 against the skin and press the inject button 258 for injection. The images of FIG. 37(a) to FIG. 37(c) are repeatedly displayed until the touch sensor 275 detects skin contact. If the touch sensor 275 then detects skin contact, the image in FIG. 37(d) is displayed, which prompts the user to press the inject button 258. While the drug is being injected, the images of FIG. 37(e) and FIG. 37(f) are repeated in sequence. If the touch sensor 275 detects separation from skin while the drug is being injected, the images of FIG. 37(a) to FIG. 37(c) may be repeatedly displayed again.

Thus, with the drug injection device 200 of the present embodiment, it is possible to provide a more user-friendly operation to the operator by using the information of the first temperature sensor, the information of the RF tag 16, and the detection signals of the acceleration sensor 276 and the touch sensor 275.

### Second Embodiment

The drug injection device of the present disclosure may be configured to directly house the drug cartridge 10 without using the cassette 100. With reference to the drawings, an embodiment of a drug injection system including a drug injection device 500 that does not use the cassette 100 will now be described.

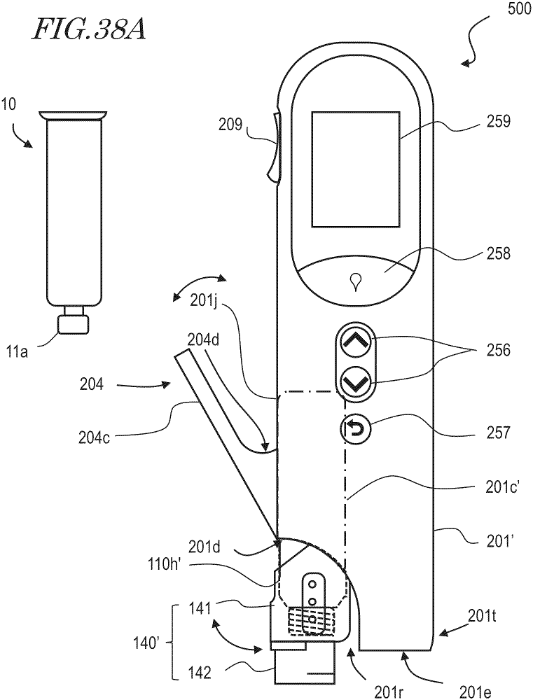

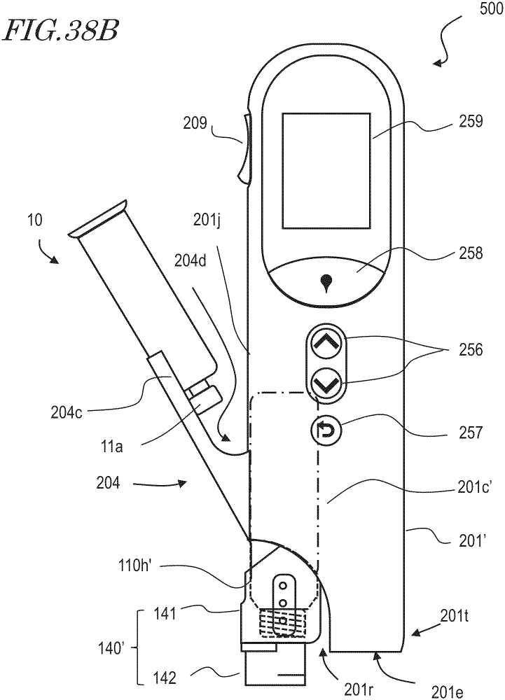

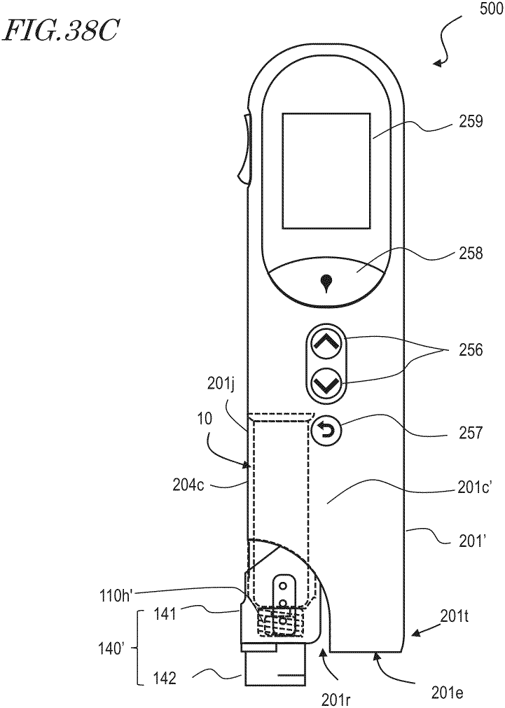

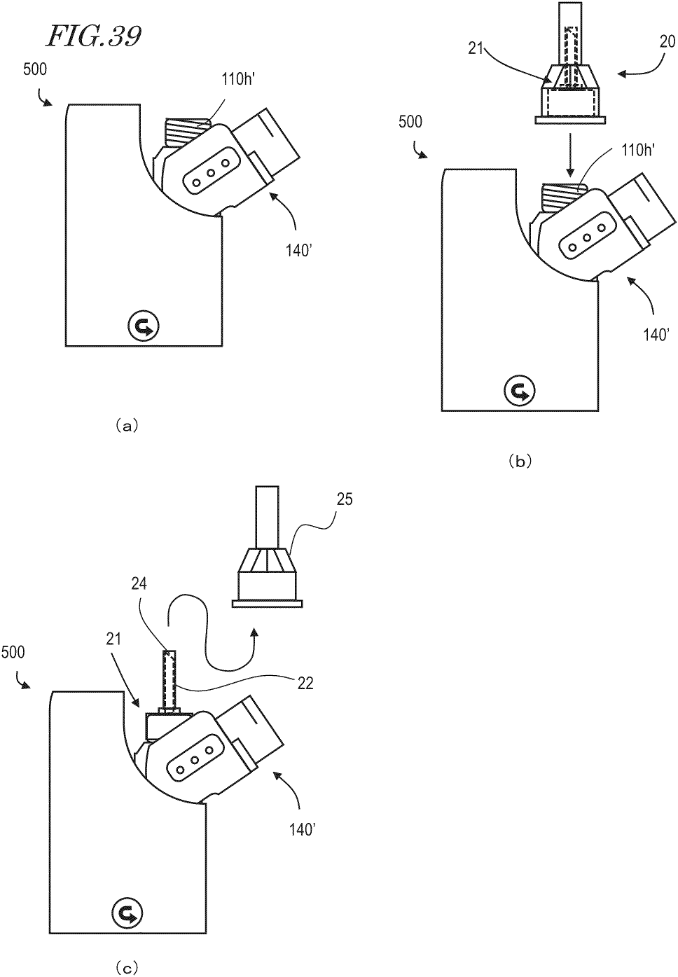

FIG. 38A to FIG. 38C are front views showing how the drug cartridge 10 is loaded in the drug injection device 500. FIG. 39 to FIG. 41 are views illustrating how a needle unit 20 is attached to, or a used injection needle 21 is removed from, a drug injection device 500 with the drug cartridge loaded. FIG. 42 is views illustrating how an injection is performed using the drug injection device 500. The structure and operation of a drug injection device 500 will be described with reference to these figures.

With the drug injection device 500, the cassette 100 with the drug cartridge 10 housed therein is not loaded in the drug injection device 500, but the drug cartridge 10 is directly loaded in the drug injection device 500. The drug injection system does not include the cassette 100. Otherwise, a drug injection device 500 has the same structure and function as the drug injection device 200 of the first embodiment. That is, a drug injection device 500 includes the RF-ID reader 277, the piston drive mechanism 220, the touch sensor 275, the acceleration sensor 276, the rotary encoder 265, etc., performs a control using these components as described above in the first embodiment, and realizes a user interface using the display device 259; these aspects remain the same also with the drug injection device 500. Therefore, the following description mainly focuses on points that differ from the first embodiment.

As shown in FIG. 38A, the drug injection device 500 includes a cartridge holder 204, a hood 140′, and an injection needle attachment portion 110h′. The drug injection device 500 also includes a housing space 201c′ in a device housing 201′ that can hold at least a portion of the drug cartridge 10 not stored in a cassette. The housing space 201c′ is adapted to house a portion of the drug cartridge 10 supported by the cartridge holder 204 described below. The device housing 201′ has a holder opening 201j connected to the housing space 201c′ . The holder opening 201j is a housing opening provided on the side surface of the device housing 201′. With the drug injection device 500, the drug cartridge 10, which is not housed in a cassette, is supported by the cartridge holder 204 and loaded through the holder opening 201j.

The cartridge holder 204 includes a door portion 204c and a holder portion 204d. The door portion 204c has a shape corresponding to the holder opening 201j so that the holder opening 201j can be blocked. The holder portion 204d has an inner space that supports the drug cartridge 10. The cartridge holder 204 is pivotably attached to the device housing 201′ so that the door portion 204c can assume a state where the holder opening 201j is open and a state where the holder opening 201j is closed. As shown in FIG. 38B, when the door portion 204c is in a state where the holder opening 201j is open, the drug cartridge 10 can be supported in the cartridge holder 204 by inserting the drug cartridge 10 into the holder portion 204d from the first end 11a. It is also possible to pull out the drug cartridge 10 supported in the holder section 204D. With the drug cartridge 10 inserted into the holder portion 204d, the cartridge holder 204 is rotated to close the holder opening 201j with the door portion 204C. As shown in FIG. 38C, with the holder opening 201j closed by the door portion 204c, a portion of the drug cartridge 10 is arranged in the housing space 201c′. The distal end portion of the drug cartridge 10, including the first end 11a, is housed in the injection needle attachment portion 110h.

The injection needle attachment portion 110h′ includes an internal space for storing the distal end portion of the drug cartridge 10, and is arranged so as to cover the housing opening 201d located at the bottom of the device recess portion 201r of the device housing 201′. The injection needle attachment portion 110h′ includes a structure similar to the injection needle attachment portion 110h of the cassette 100.

The hood 140′ has a structure similar to the hood 140 of the cassette 100 of the first embodiment, except that it does not include the arms 141c (shown in FIG. 11). The hood 140′ is located in the device recess portion 201r of the housing 201′ and is pivotally attached to the housing 201′. The hood 140′ covers the injection needle attachment portion 110h′ and can pivot with respect to the device housing 201′ in the direction indicated by the arrow in FIG. 38A. Specifically, it can pivot between the first position in which the injection needle attachment portion 110h′ is covered and the second position in which the injection needle attachment portion 110h′ is exposed. With the injection needle attachment portion 110h′ exposed from the hood 140′, the operator can easily attach the needle unit 20 to the injection needle attachment portion 110h′ and remove the needle unit 20 engaged with the injection needle attachment portion 110h′. The needle concealment 142 may be at least partially transparent as described with reference to FIG. 11.

Next, the operation of attaching the needle unit 20 to the drug injection device 500 in which the drug cartridge 10 is loaded in order to perform injection will be described. As shown in FIG. 39(a), first, the hood 140′ is pivoted to expose the injection needle attachment portion 110h′ from the hood 140′. As shown in FIG. 39(b), the needle unit 20 that houses the injection needle 21 is attached to the injection needle attachment portion 110h′. Then, as shown in FIG. 39(c), the needle case 25 is removed.

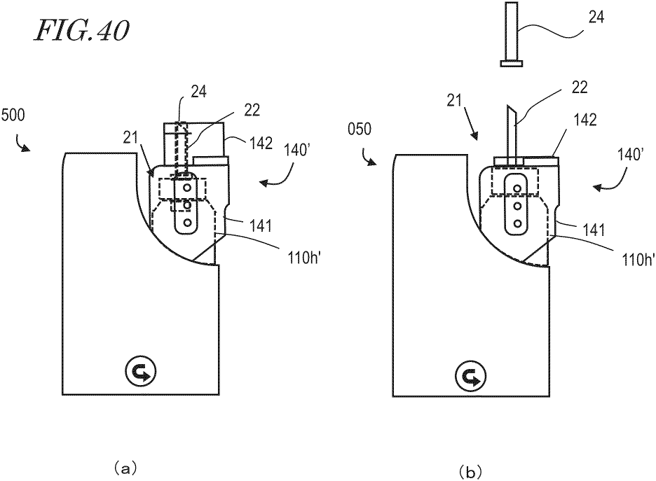

The hood 140′ is returned to its original position as shown in FIG. 40(a), and the needle covering 142 is pushed down as shown in FIG. 40(b). Then, the needle cap 24 is removed to expose the needle 22 of the injection needle 21. Then, injection can be performed.

As described in the first embodiment, when performing injection, the drug injection device 500 is held so that the distal end of the needle 22 faces down, as shown in FIG. 42(a). The needle 22 is covered by the hood body 141 and the needle cover 142. As shown in FIG. 42(b), in this state, when the skin-contact surface 201e is brought into contact with the skin 510 and the drug injection device 500 is further pressed against the skin, the needle 22 is inserted into the skin 510 while the needle covering 142 retracts. Then, the inject button 258 is pressed to inject the drug.

After completion of injection, the drug injection device 500 is pulled off the skin 510, and the needle covering 142 moves forward and covers the needle 22 as shown in FIG. 42(c).

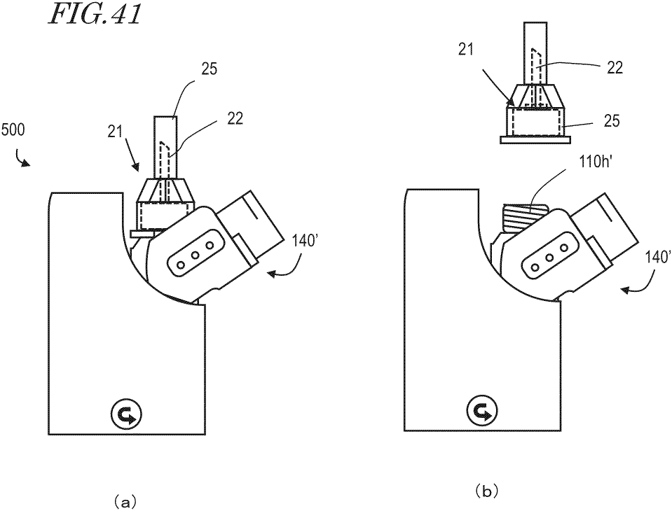

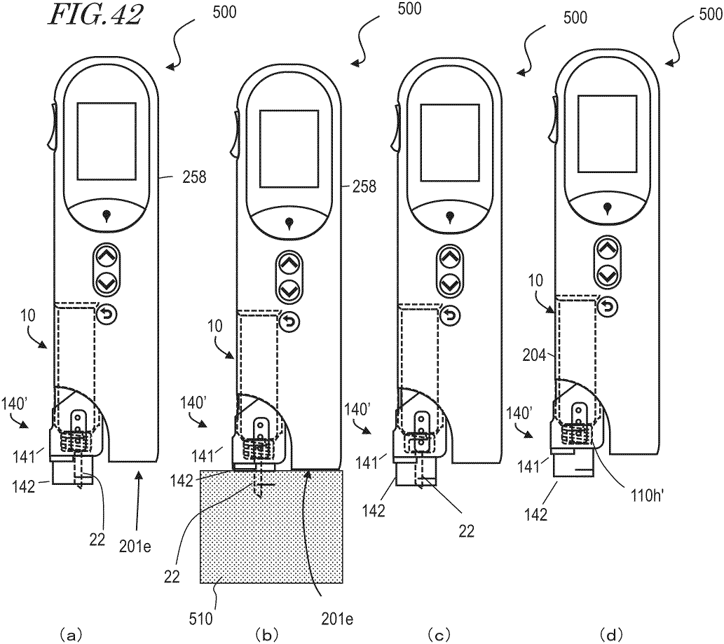

When injection is complete, the needle case 25 is placed over the injection needle 21 with the hood 140′ covering the needle 22. Then, as shown in FIG. 41(a), with the hood 140′ rotated, the needle case 25 is pulled while being rotated, so the injection needle 21, together with the needle case 25, is pulled out of the injection needle attachment portion 110h′ as shown in FIG. 41(b).

By returning the hood 140′ to the original position, the injection needle attachment portion 110h′ is covered by the hood 140′ as shown in FIG. 42(d). Then, the cartridge holder 204 is rotated and the drug cartridge 10 is taken out, thereby completing injection.

As described above, the drug injection device 500 can manage the temperature of the drug using the RF-ID reader 277, control the motor according to the type of drug using the piston drive mechanism 220, determine skin contact using the touch sensor 275, control the attitude of the drug injection device 500 using the acceleration sensor 276 and determine a fault using the rotary encoder 265 as in the first embodiment.

A charger compatible with the drug injection device 500 of the present embodiment may, for example, have a space into which the distal end of the drug injection device 500 cannot be inserted with the drug cartridge 10 loaded in the drug injection device 500. In this case, although not shown in the figures, some mechanism can be added such that the attachment of the drug cartridge 10 causes a change in the external appearance and external shape of the device housing 201′, thereby inhibiting the attachment to the charger. By inhibiting the attachment to the charger, deterioration of the drug due to heat generated during charging can be avoided.

Alternatively, the charger may have a space in which the distal end of the drug injection device 500 cannot be inserted, for example, with the needle unit 20 attached to the first end 11a of the drug cartridge 10. In addition to avoiding deterioration of the drug due to heat generated during charging, this configuration is also effective in reducing the size of the drug injection device and the charger, and in preventing infection, etc., caused by reuse of injection needles.

With the drug injection device 500 of the present embodiment, it is possible to provide operability that is easy for the operator to use as described in the first embodiment without the use of the cassette 100.

The drug injection device 500 of the present embodiment is suitable, for example, when the drug cartridge 10 houses a single dose of drug to be injected into an operator such as a patient. Since the used drug cartridge 10 is disposed of, there is no need to store it in a refrigerator or other cold place using the cassette 100. Thus, the cost of injection can be reduced. However, the drug cartridge 10 may store multiple doses of drug. In this case, a separate storage case may be provided and the drug cartridge 10 containing the remaining drug may be stored in a refrigerator, or the like, or the entire drug injection device 500 to which the drug cartridge 10 is attached may be stored in a refrigerator, or the like. However, if the entire drug injection device 500 is stored in a cold place, the reminder operation described in the first embodiment above will be difficult to perform because the drug injection device 500 itself is also cooled.

### Other Embodiments

The embodiment described above is one example of the drug injection device of the present disclosure, and various modifications can be made thereto. The shapes of the drug cartridge 10, the cassette 100, the drug injection device 200 and the charger 300 illustrated in the figures and their constituent parts are examples, and they may have other shapes. The drug injection device 200 does not need to include all of the various sensors. The injection operation, the air-removing operation, the mixing operation, etc., according to the temperature of the drug described with reference to the flow charts are examples, and some of the steps may be omitted or performed in a different order, or other steps may be included.

The estimation of the time at which the drug reaches the appropriate temperature may be performed based on a calculation other than Equations (1) to (3), or the time at which the drug reaches the appropriate temperature may be determined directly as a function of elapsed time. The fault detector 281 may include other functional blocks or may perform signal processing different from the embodiments described above to determine the fault.

### INDUSTRIAL APPLICABILITY

The cassette, the drug injection device and the drug injection system of the present disclosure can suitably be used for devices for injecting various drugs.

REFERENCE SIGNS LIST

| | |
|---|---|
| 10, 10′ | Drug cartridge |
| 11 | Cylinder |
| 11′ | Cylinder |
| 11 a | First end |
| 11 b | Second end |
| 11 c | Cylinder columnar space |
| 11 c 1 | First region |
| 11 c 2 | Second region |
| 11 c 3 | Third region |
| 11 d | Cylinder opening |
| 11 e | Bypass space |
| 11 h | Corner |
| 11 j | Shaft |
| 11 t | Projecting portion |
| 12 | Cylinder cap |
| 13 | Gasket |
| 13A | First gasket |
| 13B | Second gasket |
| 14 | Drug |
| 14A | Liquid component |
| 14B | Solid component |
| 15 | First temperature sensor |
| 16, 16′ | RF tag |
| 16 a | Antenna |
| 17 | Label |
| 20 | Needle unit |
| 20 a | Distal end |
| 20 c | Housing space |
| 20 t | Protruding portion |
| 21 | Injection needle |
| 22 | Needle |
| 23 | Connecting portion |
| 24 | Needle cap |
| 25 | Needle case |
| 31 | Charger housing |
| 100 | Cassette |
| 110 | Cassette body |
| 110 a | Distal end |
| 110 b | Rear end |
| 110 c | Cassette columnar space |
| 110 e | Body opening |
| 110h, 110h′ | Injection needle attachment portion |
| 130 | Cassette cap |
| 130 d | Cap opening |
| 140, 140′ | Hood |
| 141 | Hood body |
| 141 b | Rear end |
| 141 c | Arm |
| 142 | Needle covering |
| 142 c | First portion |
| 142 d | Second portion |
| 143 | Biasing member |
| 200 | Drug injection device |
| 200 a | Distal end section |
| 201 | Device housing |
| 201 a | Distal end section |
| 201c, 201c′ | Housing space |
| 201 d | Housing opening |
| 201 e | Skin-contact surface |
| 201 g | Charging terminal |
| 201 r | Recess portion |
| 201 t | Protruding portion |
| 202 | Internal housing |
| 202 d | Gear region |
| 202 f | Piston guide region |
| 202 g | Protruding portion |
| 202 h | Cassette region |
| 203 | First guide |
| 204 | Cartridge holder |
| 204 c | Door portion |
| 204 d | Holder portion |
| 209 | Eject lever |
| 210 | Piston |
| 210 a | First end |
| 210 b | Second end |
| 210 h | Hole |
| 211 | Distal end section |
| 212 | Body |
| 213 | Drive protruding portion |
| 214 | Female thread |
| 220 | Piston drive mechanism |
| 221 | Gearbox |
| 222 | Drive gear |
| 223 | Bearing |
| 230 | Piston guide |
| 230 g | Groove |
| 230 h | Hole |
| 231 | Guide |
| 235 | Drive rod |
| 236 | Male thread |
| 251 | Control section |
| 252 | Charging section |
| 253 | Secondary battery |
| 254 | Memory |
| 255 | Power button |
| 256 | Select button |
| 257 | OK button |
| 258 | Inject button |
| 259 | Display device |
| 260 | Buzzer |
| 261 | Clock |
| 262 | Communication section |
| 263 | Motor driver |
| 264 | Motor |
| 265 | Rotary encoder |
| 266 | Encoder plate |
| 266N | Normal blade section |
| 266Nc | Notch |
| 266Nf | Blade |
| 266S | Reference blade section |
| 266Sc | Notch |
| 266Sf | Blade |
| 267 | Pulse encoder |
| 267 c | Light-emitting element |
| 267 d | Light-receiving element |
| 270C | Transmission/reception circuit |
| 271 | Piston origin detector |
| 272 | Cassette loading detector |
| 273 | Second temperature sensor |
| 274 | Eject lever detector |
| 275 | Touch sensor |
| 276 | Acceleration sensor |
| 277 | RF-ID reader |
| 278 | Antenna |
| 278A | First portion |
| 278B | Second portion |
| 279 | Transmission/reception circuit |
| 280 | Control device |
| 281 | Fault detector |
| 282 | Blade information obtaining section |
| 283 | Breakdown detection section |
| 284 | Speed stability determination section |
| 285 | Reference blade detection section |
| 286 | Number-of-blades detection section |
| 287 | Detection section |
| 290 | Main board |
| 291 | First sub-board |
| 292 | Second sub-board |
| 300 | Charger |
| 301 | Charger housing |
| 301 d | Rib |
| 301 e | Supply terminal |
| 301 r | Charger recess portion |
| 301 s | Step |
| 301 u | Space |
| 310 | Image |
| 310a, 311a, 312a | First region |
| 310 b | Second region |
| 310 c | Third region |
| 311, 312, 313 | Image |
| 400 | Drug injection system |

1-7. (canceled)

8. A drug injection device, comprising:
a device housing having a housing space that houses at least a portion of a cassette that houses a drug cartridge having a first temperature sensor and an RF tag, or at least a portion of a drug cartridge having a first temperature sensor and an RF tag and being not housed in the cassette, and a housing opening that communicates with the housing space;
a piston supported so that the piston can move into the housing space;
a motor configured to drive the piston;
a motor driver configured to generate a drive signal for driving the motor;
an antenna arranged adjacent to the housing space;
a transmission/reception circuit configured to transmit electromagnetic waves from the antenna and receive electromagnetic waves received by the antenna;
a display device configured to output information regarding an injection operation; and
a control device configured to control the motor driver, the transmission/reception circuit and the display device, wherein:
with the drug cartridge loaded in the housing space, the control device causes the transmission/reception circuit to receive, via the antenna, drug information including information indicating a type of a drug in the drug cartridge transmitted from an RF tag of the drug cartridge and first temperature information detected by the first temperature sensor, and determines a drive power of the motor based on the first temperature information to control the motor driver so as to output the determined drive power.

9. A drug injection device, comprising:
a device housing having a housing space that houses at least a portion of a cassette that houses a drug cartridge having a first temperature sensor and an RF tag, or at least a portion of a drug cartridge having a first temperature sensor and an RF tag and being not housed in the cassette, and a housing opening that communicates with the housing space;
a piston supported so that the piston can move into the housing space;
a motor configured to drive the piston;
a motor driver configured to generate a drive signal for driving the motor;
an antenna arranged adjacent to the housing space;
a transmission/reception circuit configured to transmit electromagnetic waves from the antenna and receive electromagnetic waves received by the antenna;
a memory storing control information for controlling the motor for each of a plurality of drugs, wherein the control information of each drug includes a set of control parameters of the motor for each of a plurality of temperature ranges;
a display device configured to output information regarding an injection operation; and
a control device configured to control the motor driver, the transmission/reception circuit, the memory and the display device.

10. The drug injection device according to claim 9, wherein:
the RF tag stores drug information including information indicating at least a type of the drug;
with the drug cartridge loaded in the housing space, the control device is configured to:
cause the transmission/reception circuit to transmit a control signal via the antenna;
cause the transmission/reception circuit to receive, via the antenna, first temperature information and drug information output from the first temperature sensor and the RF tag of the drug cartridge;
determine one set of control parameters from the control information stored in the memory based on the first temperature information and the drug information; and
control the motor using the determined set of control parameters.

11. The drug injection device according to claim 10, wherein the motor driver generates a drive signal by pulse width modulation and outputs the drive signal to the motor.

12. The drug injection device according to claim 11, wherein in the control information of each drug, a duty ratio of the drive signal by the pulse width modulation of a set of control parameters for the motor is smaller as a lower limit temperature of a temperature range of the set of control parameters for the motor is higher.

13. The drug injection device according to claim 11, wherein:
in the control information of each drug, the set of control parameters of the motor for each temperature range includes an initial value and an increment of duty ratio of the drive signal by pulse width modulation; and
the control device causes the motor driver to generate the drive signal so that current flowing in the motor increase in steps.

14. The drug injection device according to claim 10, wherein the control device does not drive the motor when the first temperature information is in a first temperature range that is less than a first temperature or equal to or greater than a second temperature.

15. The drug injection device according to claim 14, wherein when the first temperature information is in a second temperature range that is equal to or greater than the first temperature and less than a third temperature lower than the second temperature, the control device controls the display device to display information indicating to wait before injection.

16. The drug injection device according to claim 15, wherein when the first temperature information is in a third temperature range that is equal to or greater than the third temperature and less than the second temperature, the control device controls the display device to display information indicating that it is possible to perform injection.

17. The drug injection device according to claim 15, further comprising a second temperature sensor provided in the device housing and configured to output second temperature information indicating a temperature inside the device housing.

18. The drug injection device according to claim 17, wherein when the second temperature information is equal to or greater than a predetermined temperature, the control device successively obtains the first temperature information at predetermined time intervals, successively calculates estimated time until it is possible to perform injection based on the first temperature information and the second temperature information, and controls the display device to display information indicating the calculated estimated time.

19. The drug injection device according to claim 8, wherein:
the antenna includes a first portion and a second portion, which can independently receive signals from outside; and
the first portion and the second portion are arranged orthogonal to each other adjacent to the housing space.

20-35. (canceled)
